(19) United States
(12) Patent Application Publication
(10) Pub. No.: US 2020/0279648 A1
(43) Pub. Date: Sep. 3, 2020

(54) Indication Method, Indication Apparatus and Design Method for Desiging the Same

(71) Applicant: Brainscan Holding B.V., Vorden (NL)

(72) Inventor: Marc Meddens, Vorden (NL)

(21) Appl. No.: 16/608,900

(22) PCT Filed: Apr. 26, 2018
(86) PCT No.: PCT/NL2018/050278

§ 371 Date: Oct. 28, 2019

(30) Foreign Application Priority Data
Apr. 28, 2017 (NL) 2018813

(51) Int. Cl. G16H 50/20; G06N 7/00; G06N 5/02

(52) U.S. Cl. CPC G16H 50/20; G06N 5/02; G06N 7/005

(57) ABSTRACT

An indication method, and indication apparatus as well as a design method for designing the same are provided. The indication method and the indication apparatus provide a combined prediction value for predicting a condition with an individual on the basis of a plurality of partial prediction values obtained from a set of values of parameters, relevant for the condition, that are determined for the individual. The design method enables a proper selection of the parameters to be used and defines an assignment of partial prediction values to parameter values.

### RELATED APPLICATIONS

This application is a National Stage Application under 35 U.S.C. 371 of co-pending PCT application PCT/NL2018/050278 designating the United States and filed Apr. 26, 2018; which claims the benefit of NL application number 2018813 and filed Apr. 28, 2017 each of which are hereby incorporated by reference in their entireties.

The present invention pertains to an indication method for providing a diagnostic indicator.

The present invention further pertains to an indication apparatus for providing a diagnostic indicator.

The present invention still further pertains to a design method for identifying parameters and defining evaluation criteria for providing a definition of an indication method and/or indication apparatus.

### BACKGROUND

For many applications it is desired to provide a diagnostic indicator, i.e. a prediction value that indicates the likelihood that a condition exists with an individual or may come into existence. For a particular application often a plurality of parameters are available that to some extent indeed may be associated with the specific condition, but that taken apart are not sufficient to provide a reliable indication for the likelihood of the presence of the condition. A diagnostic indicator is for example considered valuable for diagnosis of psychiatric disorders, for example to assist a psychiatrist in providing a proper diagnosis using biomarkers. A biomarker is defined as a characteristic that is objectively measured and evaluated as an indicator of normal biological processes, pathogenic processes, or pharmacologic responses to a therapeutic intervention. For example a variety of biomarkers is known that is considered indicative to some extent for a major depressive disorder (MDD), see PCT/NL2014/050054). However, many of those are relatively weak indicators, and often the distribution of such indicators for a case group substantially overlaps the distribution of a control group.

### SUMMARY

It is an object of the present invention to provide an indication method for providing a diagnostic indicator using the proper associated parameters according to proper evaluation criteria.

It is a further object of the present invention to provide an indication apparatus for providing a diagnostic indicator using the proper associated parameters according to proper evaluation criteria.

It is a still further object of the present invention to provide a design method that is suitable for identifying the proper parameters and defining the evaluation criteria for providing a definition of an indication method and/or indication apparatus.

In accordance with the above, a design method is claimed in claim 1. The design method is configured to identify one or more indicative parameters and to define evaluation criteria to be used by the indication method and/or indication apparatus to issue a combined prediction value for the probability of an individual having a condition based on the individual respective parameter values determined for said indicative parameters with said individual and said evaluation criteria. The design method comprises a sequence of steps that is performed for each indicative parameter in a set of parameters. This sequence of steps may be repeated for each of the parameters. Alternatively the sequence may be simultaneously applied to two or more or all parameters, for example by using a parallel processor or by crowd computing resources. The sequence of steps may be applied to parameters that have not yet been investigated. Alternatively, or in addition, the set of parameters on which the sequence of steps is applied may be obtained from earlier investigations that suggest a relationship between certain parameters and the condition. The design method involves identification of a control group and a case group by applying a Golden Standard. For entities of the first group (control group) an independent and reliable judgment has been made that it is unlikely that they have the specified condition. For entities of the second group (case group) an independent and reliable judgment has been made that it is likely that they have the specified condition. The partitioning into these two groups according to the Golden Standard may be defined for example by experts in the field, such as medical experts. Dependent on the circumstances, the wording “likely” may imply an absolute certainty of the presence of the condition or may imply that a probability that the condition is present exceeds a minimum value. The meaning of “unlikely” is complimentary thereto.

As an example of the first, the condition that is investigated may be whether or not a mother gives birth to a child having trisomy. In this case after the child has been born, the presence or absence of this condition can be determined with certainty. As an example of the second the condition that is investigated may be whether or not a person suffers from a major depressive disorder. Due to the heterogeneous nature of MDD and symptomatic overlap with other psychiatric and somatic disorders, diagnosis may be complicated. Typically the probability for a person to have this disorder is indicated by a number in the range of 0 to 100. In this case it may be decided that persons having a value for this number lower than a threshold number (e.g. 10) are not likely to have this disorder, and are assigned to the first group (control group) and that persons having a value equal to or higher than the threshold number are likely to have this disorder, and are assigned to the second group (case group).

Now for each individual in the first group a respective first value is obtained for each indicative parameter. It is noted that the assignment to one of the first group and the second group may take place at a point in time subsequent to the time of obtaining the first value. For example, for the purpose of obtaining the parameter values a mother may have partitioned in a medical investigation during her pregnancy and the assignment to the first or the second group may have taken place after birth of the child.

The first values so obtained are used to determine a first distribution for the indicative parameter.

The same steps are performed for a second group of entities that is likely to have the condition according to the Golden Standard. I.e. a respective second value for the indicative parameter is obtained with each individual in the second group, and a second distribution is obtained based on the set of second values so obtained.

In the next step a respective indicative parameter value is determined for each of the distributions at a first predetermined percentile lower than 50. For example the first predetermined percentile is 30 and the 30^{th} percentile value of the first and the second distribution are determined, i.e. the respective parameter values for which the accumulated probability (probability mass) of the two distributions is 30%.

Then it is determined which of the first and said second distribution has the higher parameter value for the first predetermined percentile, and this distribution is identified as the selected distribution.

Subsequently, a parameter value is determined at a second predetermined percentile lower than 50 for the selected distribution. The second predetermined percentile may be the same as the first predetermined percentile or may be different, e.g. 10 or 20, i.e. the parameter value where the probability mass of the selected distribution is 10% or 20% respectively.

The parameter value so obtained defines at least a first value range with that parameter value as an upper bound and a second value range with that parameter value as a lower bound.

Depending on which of the first (control) and second (case) distribution is the selected distribution, partial prediction values are assigned to the value ranges. If the selected distribution is the first distribution then a partial prediction value is assigned to the first value range that is a stronger indicator for said condition than a partial prediction value that is assigned to the second value range. If the selected distribution is the second distribution then a partial prediction value is assigned to the first value range that is a stronger indicator for the absence of said condition than a partial prediction value assigned to the second value range. Alternatively or additionally, analogous steps can be applied when considering the distributions of a third predetermined percentile higher than 50. I.e. a respective parameter value of each of the distributions is determined for the third predetermined percentile, e.g. 70 and the one having the lower parameter value for the third predetermined percentile is selected.

Subsequently, a parameter value is determined at a fourth predetermined percentile higher than 50 for the selected distribution. The fourth predetermined percentile may be the same as the first predetermined percentile or may be different, e.g. 80 or 90, i.e. the parameter value where the probability mass at the right tail of the selected distribution is 20% or 10% respectively. The parameter value so obtained defines at least a third value range with that parameter value as an upper bound and a fourth value range with that parameter value as a lower bound.

Similarly as for the first and the second value range a partial prediction value is assigned. If the selected distribution is the first distribution then a partial prediction value assigned to the fourth value range is a stronger indicator for said condition than a partial prediction value assigned to the third value range. If the selected distribution is the second distribution then a partial prediction value assigned to the fourth value range is a stronger indicator for the absence of said condition than a partial prediction value assigned to the third value range. It is noted that it is not necessary that merely a single value is assigned to each of the ranges. For example in an embodiment partial prediction values are assigned having a magnitude that decreases as a stepwise function of a probability mass of the selected distribution for a parameter value within said first and/or said second value range and or within said third and/or fourth value range. For example the magnitude may decrease stepwise in the left tail of a selected distribution from a value 7 in a range where the probability mass is less than 1% to 2 in a range where the probability mass between 1% and 5% and to 1 in a range where the probability mass is between 5% and 10%, and likewise the magnitude may decrease stepwise in the right tail of a selected distribution from a value 7 in a range where the probability mass is less than 1% to 2 in a range where the probability mass between 1% and 5% and to 1 in a range where the probability mass is between 5% and 10%. In a range between a first value and a second value where the probability mass for the selected left tail is higher and the probability mass for the selected right tail each are higher than 10% the magnitude is set to 0 The magnitude determines the extent to which the partial prediction value is indicative. If the selected distribution is the first distribution then a higher magnitude implies that the partial prediction value is a stronger indicator for the condition, whereas if the selected distribution is the second distribution then a higher magnitude implies that the partial prediction value is a stronger indicator for the absence of the condition. For example, indication of the condition may be provided in that the sign of the partial prediction value is positive and indication of the absence of the condition may be provided by a negative sign of the partial prediction. In this way the partial prediction values can be simply add to obtain the combined prediction value. Alternatively, a positive sign and a negative sign may be used to indicate the absence or the presence of the condition, provided that this convention is systematically applied for each of the parameters The magnitude so assigned accordingly determines the relative contribution for a parameter dependent on the value of the parameter. In this regard it is noted that the selected distribution that defines the first and second range is not necessarily the same as the selected distribution that defines the third and fourth range.

As an alternative, a magnitude of the assigned partial prediction values may decrease as a continuous function of the probability mass of the selected distribution for a parameter value within said first and/or said second value range and or within said third and/or fourth value range. For example the magnitude may decrease in continuous manner in the left tail of a selected distribution from a value 7 in a range where the probability mass is less than 1% to 0, for a range where the probability mass is higher than 10%. Likewise in the right tail of a selected distribution the magnitude may decrease from a value 7 in a range where the probability mass is less than 1% to a value 0 where the probability mass increases above 10%. For each of the indicative parameters, and for each of the left tail of a selected distribution and the right tail of a selected distribution a respective magnitude function may be assigned independent of the other.

The definition of the value ranges and the assigned partial prediction values determine to which extent values for said indicative parameters determined for a particular individual contribute to the combined prediction value that indicates said condition or the absence thereof with said particular individual. Alternatively or in addition a weighting may be applied that assigns a higher or lower weight of a partial prediction value for a parameter relative to the weights assigned to partial prediction values for other parameters. Also it may be contemplated to use different weightings and different assignments of the partial prediction values for the selected left tail and the selected right tail respectively. With the design method as presented above, an indication apparatus as specified below can be designed. The indication apparatus is arranged for computing a combined prediction value indicative for the likelihood of a condition with an individual. The apparatus comprises a parameter value issuing module, a partial prediction value assignment module, and a combining module.

The parameter value issuing module issues for the individual respective individual values for a set of indicative parameters indicative for said condition. Each of the indicative parameters is associated with respective parameter value ranges, including one or more of a pair of a first value range and a second value range, and a pair of a third value range and a fourth value range. The partial prediction value assignment module determines for each of the indicative parameters which of the associated value ranges comprises the individual value for that indicative parameter, and determines the partial prediction value that should be assigned to that individual value according to its associated value range. The combining module determines the combined prediction value by combining the partial prediction values obtained for each of the parameters. The indication apparatus obtained with the design method as presented above is characterized in that the pair of a first value range and a second value range, and/or the pair of a third value range and a fourth value range and their associated partial prediction values are related to the above-mentioned first distribution and to the above-mentioned second distribution in that the first value range has the parameter value of the first selected distribution for the second percentile as an upper bound and the second value range has that parameter value as a lower bound. The indication apparatus obtained with the design method as presented above is further characterized in that a partial prediction value assigned to the first value range contributes more to the combined prediction value predicting the condition than a partial prediction value assigned to the second value range if the first selected distribution is the first distribution, whereas a partial prediction value assigned to the first value range contributes more to the combined prediction value predicting the absence of the condition than a partial prediction value assigned to the second value range if the selected distribution is the second distribution. Likewise the indication apparatus so obtained is characterized in that the third value range has the parameter value of the second selected distribution as an upper bound and in that the fourth value range has that parameter value as a lower bound. Furthermore in the indication apparatus so obtained a partial prediction value assigned to the fourth value range contributes more to a combined prediction value predicting the condition than a partial prediction value assigned to the third value range if the second selected distribution is the first distribution whereas a partial prediction value assigned to the fourth value range contributes more to the combined prediction value predicting the absence of the condition than a partial prediction value assigned to the third value range if the second selected distribution is the second distribution.

Likewise, with the design method as presented above an indication method as specified below can be designed. The indication method is arranged for computing a combined prediction value indicative for the likelihood of a condition with an individual. The indication method comprises an individual parameter determining step, a range associating step, a partial prediction value assignment step and a combination step.

The individual parameter determining step involves determining respective individual parameter values for a set of indicative parameters indicative for the condition with the individual. The range associating step involves associating each of the indicative parameters with a pair of a first value range and a second value range, and/or a pair of a third value range and a fourth value range. Each of the predetermined value ranges of a parameter is associated with a partial prediction value indicating the extent to which a parameter value in that predetermined value range is indicative for said condition. In the partial prediction value assignment step it is determined for the individual for each of the parameters which of its associated predetermined value ranges comprises the determined individual parameter value for the parameter, and a partial prediction value is determined for the associated predetermined value range. The combination step provides the combined prediction value for the individual by combining the partial prediction values obtained for each of the parameters. The indication method obtained with the design method as presented above is characterized in that the pair of a first value range and a second value range, and/or the pair of a third value range and a fourth value range and their associated partial prediction values are related to the above-mentioned first distribution and to the above-mentioned second distribution in that the first value range has the parameter value of the first selected distribution for the second percentile as an upper bound and the second value range has that parameter value as a lower bound. The indication method obtained with the design method as presented above is further characterized in that a partial prediction value assigned to the first value range contributes more to the combined prediction value predicting the condition than a partial prediction value assigned to the second value range if the first selected distribution is the first distribution, whereas a partial prediction value assigned to the first value range contributes more to the combined prediction value predicting the absence of the condition than a partial prediction value assigned to the second value range if the selected distribution is the second distribution. Likewise the indication method so obtained is characterized in that the third value range has the parameter value of the second selected distribution as an upper bound and in that the fourth value range has that parameter value as a lower bound. Furthermore in the indication method so obtained a partial prediction value assigned to the fourth value range contributes more to a combined prediction value predicting the condition than a partial prediction value assigned to the third value range if the second selected distribution is the first distribution whereas a partial prediction value assigned to the fourth value range contributes more to the combined prediction value predicting the absence of the condition than a partial prediction value assigned to the third value range if the second selected distribution is the second distribution.

An indication method and/or an indication apparatus according to the present invention can provide an indication for the presence of a condition of an individual with a relatively high quality even if the available parameters are only weakly indicative. A design method according to the present invention makes it possible to design such an indication method or indication apparatus for various applications in systematic and efficient manner.

### BRIEF DESCRIPTION OF THE DRAWINGS

These and other aspects are described in more detail with reference to the drawings. Therein:

FIG. 1 schematically illustrates an embodiment of a design method according to the present invention,

FIG. 2A-2J show example distributions of a biomarker,

FIG. 3 shows an aspect of an embodiment of the design method of FIG. 1,

FIG. 4 shows embodiments of a design apparatus and indication apparatus according to the present invention,

FIG. 5A, 5B shows ROC-curves for results obtained in a first application,

FIG. 6A, 6B illustrates results obtained with the aspect of the method described with reference to FIG. 3, in a second application; Therein FIG. 6A, 6B respectively illustrate a number of remaining biomarkers and a value of a quality measure as a function of the value of the selection criterion,

FIGS. 7A, 7B and 7C respectively illustrate the statistical significance of the quality measure, for three sets of parameters, in the second application,

FIG. 8 illustrates the ROC-curves for these three sets of parameters in the second application.

### DETAILED DESCRIPTION OF EMBODIMENTS

Like reference symbols in the various drawings indicate like elements unless otherwise indicated.

### Design Method

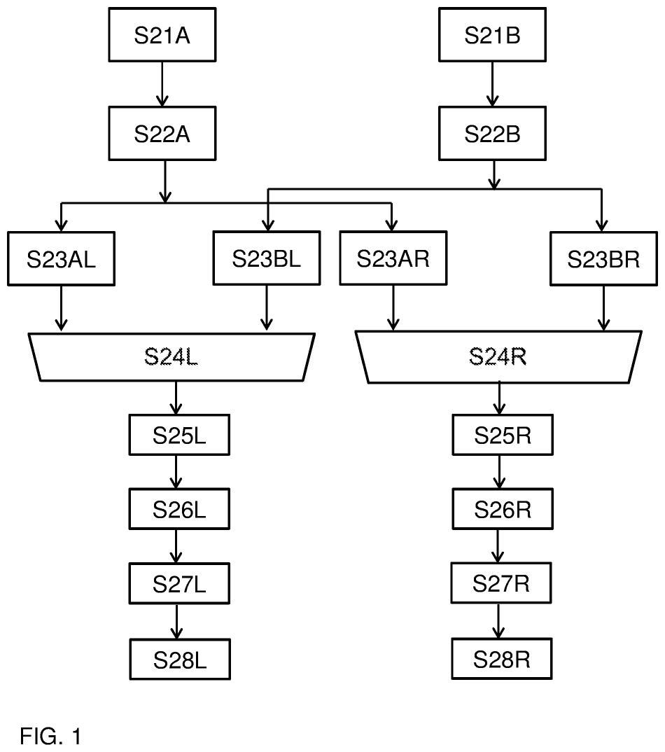

An example of a design method for providing a definition of an indication method and/or indication apparatus, as claimed is schematically shown in FIG. 1. The design method is configured to identify one or more indicative parameters and to define evaluation criteria to be used by the indication method and/or indication apparatus to issue a combined prediction value for the probability of an individual having a condition based on the individual respective parameter values determined for said indicative parameters with said individual and said evaluation criteria. As illustrated in FIG. 1, the design method comprises the following steps that are applied for each indicative parameter in a set of indicative parameters.

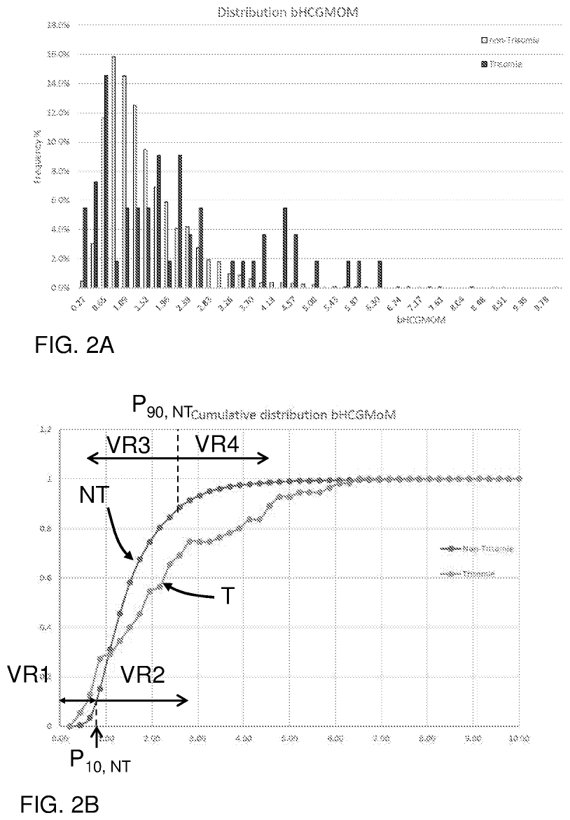

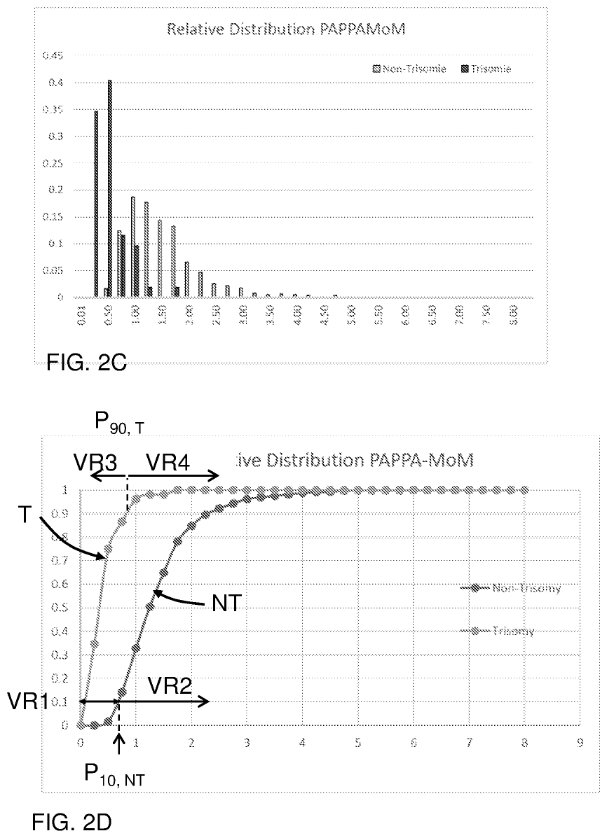

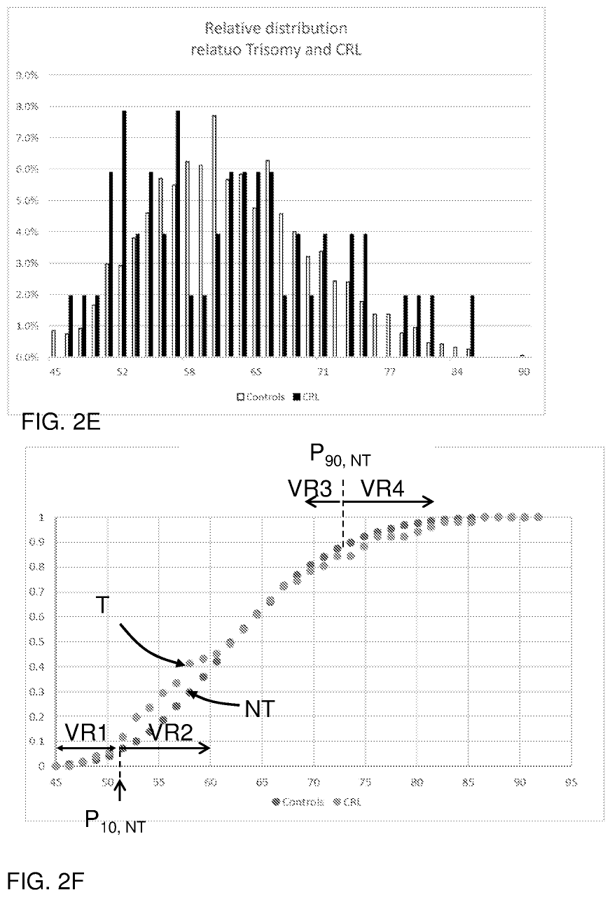

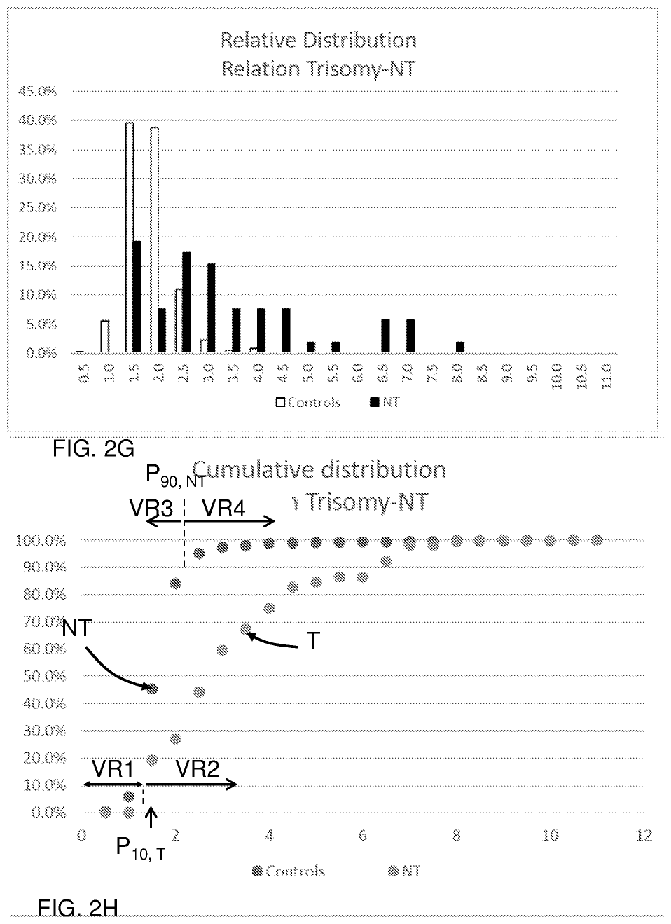

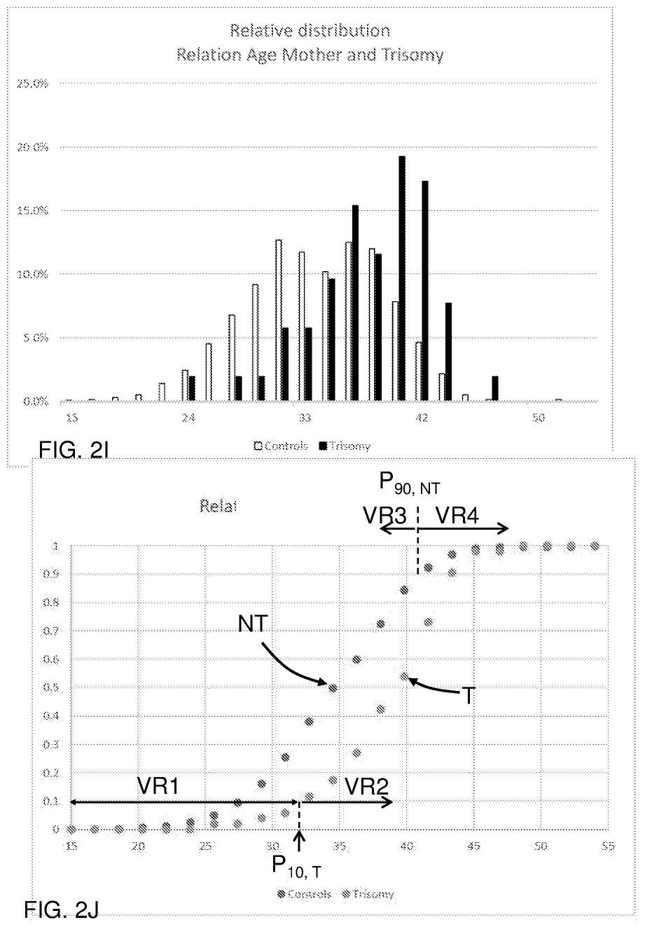

In a first step S21A a respective first value is obtained for the indicative parameter for a first group of entities that according to a Golden Standard are not likely to have the condition. In step S22A a first distribution is determined of the first values so obtained. Likewise, a distribution of second values is obtained for a second group of entities, that according to the Golden Standard are likely to have the condition. By way of example, FIG. 2A shows for each of the first and the second group the relative distribution of a parameter, here by way of example the parameter bHCGMoM which is considered indicative for the probability of giving birth to a child having trisomy. The light bars in the graph represent the first (control) distribution of the control group, and the dark bars indicate the second (case) distribution obtained with the second group of entities. Also in FIG. 2B the corresponding cumulative distributions, NT for the control group and T for the case group are shown. In practice, the set of indicative parameters may include in addition to or instead of the parameter bHCGMoM, various other indicative parameters, such as the age of the mother, weight of the mother, Parida, Gravida, Gravida minus Parida, bHCG, PAPPA, PAPPAMoM, CRL and NT.

In steps S23AL, S23BL shown in FIG. 1, respective indicative parameter values (P_{10,NT}, P_{10,T}) are determined for each of the distributions for a first predetermined percentile lower than 50. In step S24L the one of the first and the second distribution is selected that has the higher parameter value for the first predetermined percentile. In this example the first distribution NT is selected as it has the higher value (0.51) for the first percentile. In step S25L a parameter value of the selected distribution is associated with at least a second predetermined percentile lower than 50. In this case the second predetermined percentile is the same as the first predetermined percentile. Accordingly the parameter value defined in step S25L is 0.51. Therewith in step S26 at least a first value range VR1 is defined having said parameter value as an upper bound and a second value range VR2 having said parameter value P_{10,NT} as a lower bound. In step S27L a partial prediction value PP₁, PP₂ is assigned to the value ranges. In this case the selected distribution is the first distribution NT, and therewith a partial prediction value PP₁ assigned to the first value range VR1 is a stronger indicator for the condition than a partial prediction value PP₂ assigned to the second value range VR2. Similarly, in steps S23AR, S23BR a respective parameter value P_{90,NT}, P_{90,T} for a third predetermined percentile, e.g. 90, higher than 50; is determined for each of said distributions. In step S24R a distribution is selected from the first and the second distribution that has the lower parameter value P_{90,NT} for the third predetermined percentile. Also in this case the selected distribution is the distribution NT, having the lower parameter value 2.20. For this distribution NT a parameter value (P_{90,NT}) is associated with at least a fourth predetermined percentile. In this case the fourth predetermined percentile and hence the associated parameter value is 2.32. This value is used in step S26R to define at least a third value range VR3 having that parameter value as an upper bound and a fourth value range VR4 having that parameter value as a lower bound. In step S27R a partial prediction value PP₃, PP₄ is assigned to these value ranges. As the selected distribution is the first distribution NT a partial prediction value PP₄ assigned to the fourth value range VR4 is a stronger indicator for the condition than a partial prediction value PP₃ assigned to the third value range VR3.

The procedure as described above is similarly applied to the other parameters in the set of parameters. A further example thereof is shown in FIG. 2C, 2D for the parameter PAPPAMoM, In this further example the first distribution NT is selected as it has the higher value (0.42) for the first percentile. Selecting the second predetermined percentile to be the same as the first predetermined percentile a first value range VR1 and a second value range VR2 are defined having this value as an upper bound and as a lower bound respectively. As the selected distribution is the first distribution NT, a partial prediction value PP₁ assigned to the first value range VR1 is a stronger indicator for the condition than a partial prediction value PP₂ assigned to the second value range VR2. For this parameter, PAPPAMoM, the distribution T has the lower parameter value for the third predetermined percentile, i.e. P_{90,NT}=0.88. This parameter value is used to define the upper bound of the third value range and the lower bound of the fourth value range VR4. As the selected distribution is the second distribution T a partial prediction value PP₃ assigned to the third value range VR3 is a stronger indicator for the condition than a partial prediction value PP₄ assigned to the fourth value range VR3.

FIG. 2E, 2F show by way of a further example the first and the second distribution NT, T for the parameter CRL, In this further example the first distribution NT is selected as it has the higher value (52.0) for the first percentile. Selecting the second predetermined percentile to be the same as the first predetermined percentile a first value range VR1 and a second value range VR2 are defined having this value as an upper bound and as a lower bound respectively. As the selected distribution is the first distribution NT, a partial prediction value PP₁ assigned to the first value range VR1 is a stronger indicator for the condition than a partial prediction value PP₂ assigned to the second value range VR2. For this parameter, CRL, the distribution NT has the lower parameter value for the third predetermined percentile, i.e. P_{90,NT}=72.9. This parameter value is used to define the upper bound of the third value range and the lower bound of the fourth value range VR4. As the selected distribution is the first distribution NT, a partial prediction value PP₄ assigned to the fourth value range VR4 is a stronger indicator for the condition than a partial prediction value PP3 assigned to the third value range VR3. As discussed below, as a result of a further selection step, the partial prediction value determined on the basis of the parameter value CRL does not contribute in determining the combined prediction indicator.

As another example FIG. 2G, 2H show the first and the second distribution NT, T for the parameter NT, In this further example the second distribution T is selected as it has the higher value (1.25) for the first percentile. Selecting the second predetermined percentile to be the same as the first predetermined percentile a first value range VR1 and a second value range VR2 are defined having this value as an upper bound and as a lower bound respectively. As the selected distribution is the second distribution T, a partial prediction value PP₂ assigned to the second value range VR2 is a stronger indicator for the condition than a partial prediction value PP₁ assigned to the first value range VR1. For this parameter NT, the distribution NT has the lower parameter value for the third predetermined percentile, i.e. P_{90,NT}=2.20. This parameter value is used to define the upper bound of the third value range and the lower bound of the fourth value range VR4. As the selected distribution is the first distribution NT a partial prediction value PP₄ assigned to the fourth value range VR4 is a stronger indicator for the condition than a partial prediction value PP₃ assigned to the third value range VR3.

As a still further example FIG. 2I, 2J show the first and the second distribution NT, T for the parameter Age Mother, i.e. In this further example the second distribution T is selected as it has the higher value (30.9) for the first percentile. Selecting the second predetermined percentile to be the same as the first predetermined percentile a first value range VR1 and a second value range VR2 are defined having this value as an upper bound and as a lower bound respectively. As the selected distribution is the second distribution T, a partial prediction value PP₂ assigned to the second value range VR2 is a stronger indicator for the condition than a partial prediction value PP₁ assigned to the first value range VR1. For this parameter NT, the distribution NT has the lower parameter value for the third predetermined percentile, i.e. P_{90,NT}=39.4. This parameter value is used to define the upper bound of the third value range and the lower bound of the fourth value range VR4. As the selected distribution is the first distribution NT a partial prediction value PP₄ assigned to the fourth value range VR4 is a stronger indicator for the condition than a partial prediction value PP₃ assigned to the third value range VR3.

In addition, partial prediction values are assigned having a magnitude that decreases as a stepwise function of a probability mass of the selected distribution for a parameter value within said first and/or said second value range and or within said third and/or fourth value range. In this design example the magnitude decreases stepwise in the left tail of a selected distribution from a value 7 in a range where the probability mass is less than 1% to 2 in a range where the probability mass is between 1% and 5% and to 1 in a range where the probability mass is between 5% and 10%, and likewise the magnitude decreases stepwise in the right tail of a selected distribution from a value 7 in a range where the probability mass is less than 1% to 2 in a range where the probability mass between 1% and 5% and to 1 in a range where the probability mass is between 5% and 10%. In the neutral range, between 10% and 90%, the magnitude is set to 0 (zero). The results so obtained are presented in tables 1 and 2. Therein table 1 specifies for various parameters their associated value ranges and table 2 specifies the partial prediction values assigned to these parameters in these associated value ranges.

In a further step S28L, S28R, an additional selection is made as follows. To include a tail for a biomarker, it is required that the tail of the dominant group should extend substantially beyond the non-dominant tail. For instance, the probability mass of the left (or right) tail of the dominant group is required to be at least 20% at the cut-off P10 (P90) of the non-dominant group. Hence, the dominant left tail is the left tail of the non-selected distribution on the left hand side. Likewise, Hence, the dominant right tail is the right tail of the non-selected distribution on the right hand side. In addition, it is required that the probability mass of the dominant group at the left (right) of the cut-off P5 (P95) is more than 15%. If one biomarker tail does not reach these two pre-set tail criteria, the partial predication value for participants with biomarker values in these tails are set to zero. If both tails of a biomarker fail to satisfy these tail criteria, the biomarker does not contribute to disease prediction. Based on these criteria, the biomarkers “Length” and “CRL” were assigned a weight w=0, as indicated in table 1 below. The remaining biomarkers are assigned a weight w=1.

TABLE 1
Example of assigned value ranges for the condition trisomy.

| | | Percentile of selected distribution | | | | | | |
|---|---|---|---|---|---|---|---|---|
| | | P_{1, S} | P_{5, S} | P_{10, S} | N | P_{90, S} | P_{95, S} | P_{99, S} |
| | | M | | | | | | |
| Parameter | W | 7/−7 | 2/−2 | 1/−1 | 0 | 1/−1 | 2/−2 | 7/−7 |
| Age Mother (Yr) | 1 | 23.5 | 28.5 | 30.9 | | 39.4 | 40.9 | 45.4 |
| Weight (kg) | 1 | Excl | Excl | Excl | | 74.0 | 75.0 | 85.0 |
| Length (cm) | 0 | 151 | 158 | 161 | | 174 | 175 | 178 |
| Parida | 1 | Excl | Excl | Excl | | 1 | 2 | 3 |
| Gravida | 1 | Excl | Excl | Excl | | 3 | 4 | 7 |
| Gravida − Parida | 1 | Excl | Excl | Excl | | 2 | 3 | 5 |
| bhCG | 1 | 0 | 0 | 20 | | 125 | 159 | 298 |
| bhCGMoM | 1 | 0.25 | 0.43 | 0.51 | | 2.32 | 2.91 | 4.86 |
| PAPPA | 1 | 0.0 | 0.0 | 0.25 | | 1.37 | 3.53 | 3.71 |
| PAPPAMoM | 1 | 0.19 | 0.33 | 0.42 | | 0.88 | 0.96 | 1.80 |
| NT | 1 | 1.01 | 1.17 | 1.25 | | 2.20 | 2.50 | 3.90 |
| CRL | 0 | 46.5 | 50.2 | 52.0 | | 72.9 | 74.5 | 83.4 |

TABLE 2
Example of partial prediction values associated with a set of
parameters in 7 percentile ranges for the condition trisomy.

| | Percentile of selected distribution | | | | | | |
|---|---|---|---|---|---|---|---|
| Parameter | P_{1, S} | P_{5, S} | P_{10, S} | N | P_{90, S} | P_{95, S} | P_{99, S} |
| Age Mother (Yr) | −7 | −2 | −1 | 0 | 1 | 2 | 7 |
| Weight | 0 | 0 | 0 | 0 | −1 | −2 | −7 |
| Length (cm) | 0 | 0 | 0 | 0 | 0 | 0 | 0 |
| Parity | 0 | 0 | 0 | 0 | 1 | 2 | 7 |
| Gravidity | 0 | 0 | 0 | 0 | 1 | 2 | 7 |
| Gravida − Parida | 0 | 0 | 0 | 0 | 1 | 2 | 7 |
| bhCG | −7 | −2 | −1 | 0 | 1 | 2 | 7 |
| bhCGMoM | 7 | 2 | 1 | 0 | 1 | 2 | 7 |
| PAPPA | 7 | 2 | 1 | 0 | −1 | −2 | −7 |
| PAPPAMoM | 7 | 2 | 1 | 0 | −1 | −2 | −7 |
| NT | −7 | −2 | −1 | 0 | 1 | 2 | 7 |
| CRL | 0 | 0 | 0 | 0 | 0 | 0 | 0 |

In tables 1,2, the first column specifies a set of parameters that potentially could be indicative for the presence or absence of the condition, here trisomy. The second column in table 1 (not present in table 2) indicates a weight W that is assigned to the parameter. In this case the weight is either 1 or 0. A weight 1 implies that the value of the parameter is taken into account for computing the combined prediction value. A weight 0 implies that it is not taken into account for computing the combined prediction value. In other embodiments weights W other than 0 or 1 may be assigned, wherein the weight for a parameter indicates the degree to which its value is taken into account for computing the combined prediction value, and in the first row the magnitude M of the associated partial prediction value. In this example the value W=0 for the parameters “Length” and “CRL” indicates that these parameters do not contribute at all in determining the combined prediction value. Alternatively, separate weights W_{L}, W_{R} may be provided for the left tail and the right tail of a distribution respectively, to indicate whether or not that left or right tail contributes to determining the combined prediction value.

In table 1, the next columns indicate the value of the selected distribution for each of the predetermined percentiles P_{1,S}; P_{5,S}; P_{10,S}; P_{90,S}; P_{95,S}; P_{99,S}, and table 2 indicates the associated partial prediction values. For example for the parameter “Age Mother”, the respective values for the selected distribution are: 23.5; 28.5; 30.9; 39.4; 40.9 and 45.4, and the magnitudes of the associated weights are 7,2,1,(0 for the neutral range), 1,2,7. As indicated by the underlining, and as shown explicitly in table 2, the associated partial prediction values are −7, −2, −1, (0 for the neutral range), 1,2,7. As indicated above, in those cases where the probability mass of the non-selected distribution does not exceed a threshold, a parameter only partly contributes in determining the combined prediction value by excluding the tail where the threshold is not met. In table 1 this is indicated as “Excl” and in table 2, the value 0 (zero) is assigned. This also applies to the neutral range “N”, extending here between the value for P_{10,S} of the distribution selected on the left side and the value for P_{90,S} of the distribution selected on the right side.

In Table 1 the underlined data indicates the regions where a parameter value is indicative for the likelihood of the condition trisomy, and wherein the associated sign of the parameter value is positive. The non-underlined data indicates the regions where a parameter value is indicative for the likelihood of the absence of the condition trisomy and wherein the associated sign of the parameter value is negative. In this example the combined prediction value is determined by adding the individual partial prediction values. Hence, a combined prediction value having a larger value is more indicative for the likelihood of the condition trisomy then a smaller combined prediction value. Hence, the combined prediction value is more indicative for the likelihood of the condition trisomy then each of the partial prediction values and only one new parameter is obtained for interpretation of that said likelihood. In this example, arbitrarily, this combined prediction value is assigned “Trisomy Index” or abbreviated “TI”.

Returning to the example of the parameter bHCGMoM, it can be seen in table 1 that the magnitude of the assigned partial predication value is 7 for the range 0-0.25 (P_{NT,1}), 2 in the range 0.25-0.43 (P_{NT,5}), and 1 in the range 0.43-0.51 (P_{NT,10}). As indicated by the underlining the likelihood of trisomy is indicated, i.e. the partial prediction value is positive. It can be further seen, that in the neutral range between P_{NT,10} and P_{NT,90} the partial prediction value is zero (0). It can further be seen that the magnitude of the assigned value is 7 for the range above 4.86 (P_{NT,99}), 2 in the range 2.91(P_{NT,95})-4.86, and 1 in the range 2.32(P_{NT,95})-2.91. In this case the partial prediction value contributes to an indication of the likelihood of trisomy in both tails.

Similarly other ranges are defined as shown in table 1. Whereas for the case of the parameter bHCGMoM the first distribution in both tails is the selected distribution, this is different for example for the parameter “Age Mother”. In that example the second distribution is selected for the left tail and the first distribution is selected for the right tail.

It is noted that the selected distribution for the left tail, i.e. the percentiles P_{1,S}; P_{5,S}; P_{10,S} is not necessarily the same as the one for the right tail, i.e. the percentiles P_{90,S}; P_{95,S}; P_{99,S}. In the example of the parameter “Age Mother”, the selected distribution for the left tail is the distribution of the parameter value for the cases, whereas the selected distribution for the right tail is the distribution of the parameter value for the control group. This can be seen in table 1 in that the numbers 39.4; 40.9 and 45.4, are underlined and hence are associated with a positive partial prediction value (see table 2) that contributes to a combined prediction value that indicates the condition trisomy. The numbers 23.5; 28.5; 30.9 in the left tail are not underlined and hence are associated with a negative partial prediction value (see table 2) that contributes to a combined prediction value that indicates the absence of the condition trisomy.

The definition of the value ranges and the assigned partial prediction values so obtained determine to which extent values for said indicative parameters determined for a particular individual contribute to the combined prediction value that indicates said condition or the absence thereof with said particular individual.

Therewith even with a set of relatively weakly indicative parameters it becomes possible to design an indication method or indication apparatus that provides an indication for the presence of a condition of an individual with a relatively high quality.

### Selecting Parameters

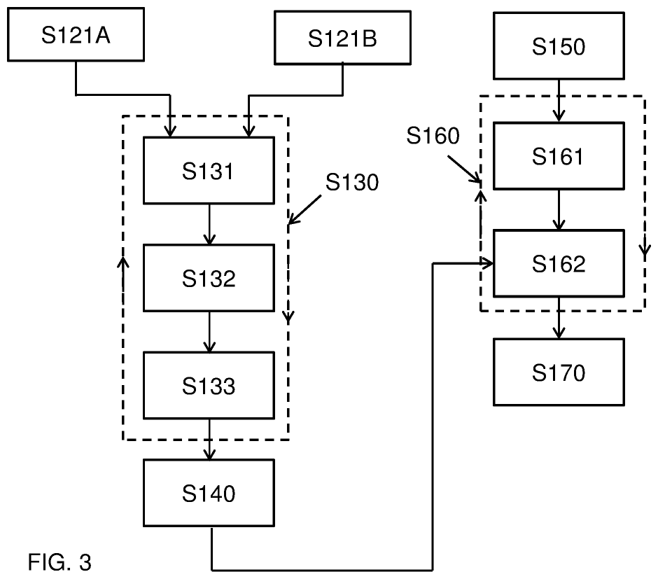

A set of parameters to be used for determining a combined prediction value may be selected from a superset with a method as schematically shown in FIG. 3. A first and second group of entities are identified, wherein the first group of entities is a group of entities that according to a Golden Standard do not have a certain condition, e.g. a depressive disorder, or are not likely to have said condition, and wherein the second group of entities is a group of entities that according to said Golden Standard have said condition or are likely to have said condition. For each parameter in the superset a first parameter value distribution is determined (S121A) for the first group of entities, and a second parameter value distribution is determined (S121B) for the second group of entities.

Having obtained these data, the following verification procedure S130 is repeated.

In a first step S131 of the verification procedure S130, for each individual in said first and said second group of entities a vector of parameter values is randomly assigned to one of a first and a second auxiliary parameter value distribution that will serve as a replacement for the first and the second parameter value distribution in the verification procedure. A vector of parameter values is defined here as the set of parameter values determined for the superset of parameters with said individual. For example the vector of parameters determined for an individual of the first group may be randomly assigned to one of the first and the second auxiliary parameter value distributions and likewise the vector of parameters determined for an individual of the second group may be randomly assigned to one of the first and the second auxiliary parameter value distributions.

In the second step S132 of this verification procedure S130 for a plurality of entities a combined prediction value is determined based on their parameter values. The plurality of entities may be entities other than those of the first and the second group, but alternatively they may be within the first and the second group. However, instead of using the first parameter value distribution and the second parameter value distribution to determine the partial prediction values the first parameter value distribution and the second parameter value distribution are replaced by the first auxiliary parameter value distribution and the second auxiliary parameter value distribution.

Using these auxiliary distributions, for each of these plurality of entities a combined prediction value may be calculated as if these auxiliary distributions were the actual first parameter value distribution and the second parameter value distribution.

In the third step S133 of the verification procedure S130 a value of a quality measure is calculated that indicates the extent to which the combined prediction value obtained for each of the entities with the superset of parameters indicates the presence of the condition according to the Golden Standard. The quality measure may be based on the determined amount of true positives (TP), false positives (FP), true negatives (TN), and false negatives (FN). An example of a quality measure is the AUC (area under the curve). This is the area under the curve defined by the relationship between the inverse of the specificity (1-specificity) and the sensitivity. The sensitivity (also called the true positive rate, the recall, or probability of detection measures the proportion of positives that are correctly identified (TP) as such (i.e. the percentage of entities who are correctly identified as having the condition). The specificity (also called the true negative rate) measures the proportion of negatives that are correctly identified (TN) as such (e.g., the percentage of entities who are correctly identified as not having the condition). An alternative quality measure is the F-measure, which is the harmonic mean between precision and recall or the F_{β}-measure, which is the weighted harmonic mean between precision and recall, i.e.

F_{β}=TP*(1+β)²/(TP*(1+β)²+FN*β²+FP)

When the verification procedure S130 has been repeated a sufficient number of times, for example a predetermined number e.g 100, 1000 or 10000, a distribution is determined in step S140 of the values obtained for the quality measure obtained with the verification procedure As the auxiliary distributions are obtained by the randomization in step S131, the distribution of the quality measure so obtained is indicative for the likelihood that a particular quality measure would be obtained with these parameters by chance. The distribution of the AUC so obtained will be centered around the value 0.5.

In step S150 mutually different candidate sets of parameters are defined within the superset of parameters. These candidate sets are obtained as subsets from the superset by applying respectively different selection criteria. The respectively different selection criteria may be subsequently more strong selection criteria. For each candidate set of the mutually different sets of parameters a procedure S160 is performed.

A quality measure is determined in step S161 that indicates to which extent the combined prediction values obtained for the plurality of entities based on the observed values for the parameters in the candidate set complies with the indication according to the Golden Standard for these entities.

In step S162 a statistical significance is determined of the quality measure based on the distribution obtained in step S140.

Then in step S170 an optimal set is selected from said mutually different candidate sets of parameters that optimizes an optimization criterion based on optimal performance (e.g. a highest AUC) or said statistical significance. The selection in step S170 may involve selecting from said candidate sets the one having the best value for said optimal performance or statistical significance. Alternatively a candidate set may be selected that with a modest number of parameters performs sufficiently, even if the optimal performance or the statistical significance among is not the highest among the other candidate sets. Also the optimization criterion may include a weight factor indicative for the ease with which a value for a parameter value can be obtained.

Typical selection criteria for identifying parameters for a candidate set may be based on differences in probability mass between the selected distribution and the non-selected distribution at a predetermined parameter value of the selected distribution for a parameter. The predetermined value for the parameter may for example be the parameter value of a fifth predetermined percentile for the selected distribution.

A candidate set may be selected by requiring that the probability mass of the non-selected distribution at a parameter value is at least a first predetermined multiplication factor times the probability mass of the selected distribution for that parameter value. For example, if the multiplication factor is 2, then the probability mass of the non-selected distribution should be at least twice as high as the probability mass of the selected distribution for a particular parameter value. E.g. if the particular parameter value is the 10^{th} percentile (P₁₀) of the selected distribution then the probability mass for the non-selected distribution at that particular parameter value should be at least 20%. Subsequently smaller candidate sets may be defined by increasing the multiplication factor. Similarly selections can be made by suitable choices of a multiplication factor for a second probability mass at a right tail of the distributions.

### Example 1: Trisomy

The procedures introduced above are now described in more detail. In this case the design procedure was applied to a clinical research on trisomy involving a total of 3285 pregnant women. The data was subdivided into a training set and a validation set of substantially the same size. The training set includes 1643 individuals of which 24 positive cases. I.e. in these 24 cases the child born after pregnancy received a Trisomy-related diagnosis. The validation set includes 1642 individuals of which 25 positive cases. Specifics are indicated in table 3 below.

TABLE 3
Specifics of the training set and the validation set

| | Training set | Validation set |
|---|---|---|
| | (n = 1643) | (n = 1642) |
| trisomy13 | 1 | 1 |
| trisomy18 | 6 | 6 |
| trisomy21 | 14 | 16 |
| 47XXX | 1 | 0 |
| triploidy | 0 | 1 |
| suspected trisomy | 1 | 0 |
| chromosomal disorder | 1 | 1 |
| Total | 24 | 25 |

Table 4 below specifies the selection criteria used to identify the subsets in the super set of parameters using the procedure of FIG. 3.

TABLE 4
Phenotype randomisation results.

| Limits | n-bm | AUC-real | AUC-random | n-rand | significance |
|---|---|---|---|---|---|
| 50/25 | 4/12 | 0.877 | 0.514 | 519 | p < 0.0001 |
| 40/20 | 6/12 | 0.878 | 0.541 | 317 | p < 0.0001 |
| 40/15 | 8/12 | 0.887 | 0.598 | 320 | p < 0.0001 |
| 30/15 | 9/12 | 0.896 | 0.607 | 315 | p < 0.0001 |
| 20/10 | 11/12 | 0.893 | 0.679 | 321 | p < 0.0001 |

The first column indicates the threshold value for the parameter values corresponding to the 10^{th} and the 5^{th} percentile of the selected distribution respectively and the threshold value for the parameter values corresponding to the 90^{th} and the 95^{th} percentile of the selected distribution, respectively. E.g. the limits in the first row specify that for these percentiles the probability mass of the non-selected distribution of a parameter should be at least 50 and 25% respectively. The second column indicates the number of remaining parameters out of the 12 original parameters. E.g. for only 4 of the 12 parameters has non-selected distribution with a probability mass of at least 50 and 25% for the parameter values corresponding to the 10^{th} and the 5^{th} percentile of the selected distribution respectively. The third column indicates the area under the curve AUC that is obtained with the selected set of parameters. The fourth column indicates the mean value of the area under the curve AUC that would be obtained with the selected set of parameters if the distributions for the cases and the controls were obtained by an arbitrary classification of the parameters in steps S130, wherein the fifth column indicates the number of repetitions n-rand of the loop S130. The sixth column indicates that the AUC values in the third column in each case are significant. I.e. the probability that the observed value for AUC-real occurs by coincidence is less than 0.0001 in each case.

The conditions 20/10 till 30/15 using 9 to 11 biomarkers appear to be optimal, as is read from the obtained AUC values. Based on the above, the following biomarkers are selected as the parameters that contribute to the combined prediction value: Age of the mother, Weight of mother, Parida, Gravida, Gravida minus Parida, bHCG, bHCGMoM, PAPPA, PAPPAMoM and NT. The Length of the mother as well as the biomarker CRL did not contribute, as indicated by the weight 0 assigned to these parameters in Table 1, and as also becomes apparent from table 2. In addition, the left tails of the parameters Weight, Parida, Gravida and Parida minus Gravida do not contribute.

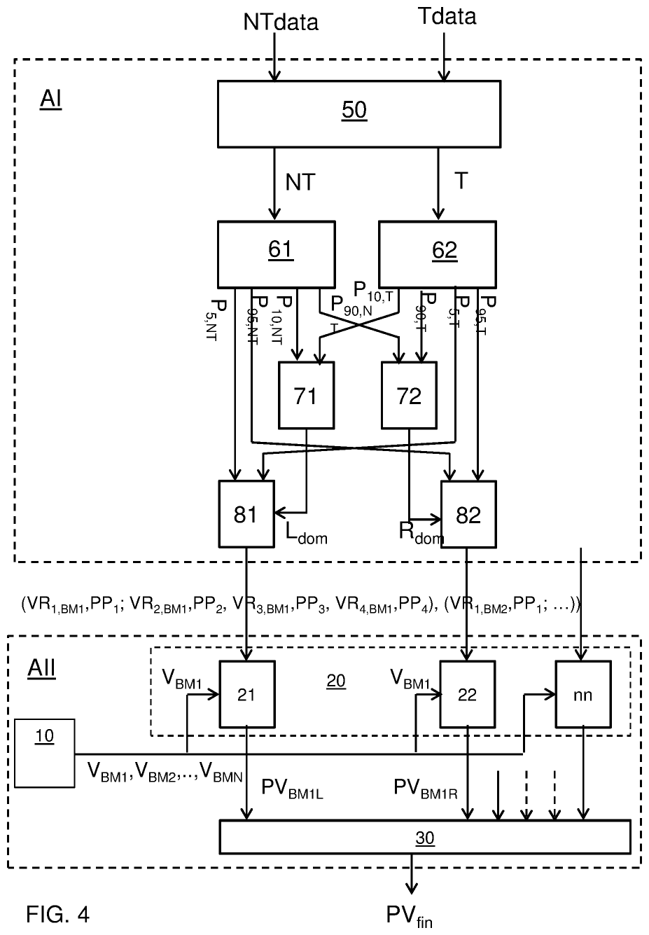

FIG. 4 schematically shows a design apparatus AI that can be used to perform the design method of FIG. 1, and illustrates how the data so obtained can be used in an efficient manner by an indication apparatus AII to determine a combined prediction value PV_{fin}, based on the value range, and partial prediction value data (VR_{1,BM1},PP₁; VR_{2,BM1},PP₂, VR_{3,BM1},PP₃, VR_{4,BM1},PP₄), (VR_{1,BM2},PP₁; . . . )) as obtained by the design method. The design apparatus AI of FIG. 4 may include a distribution composing module 50 that for each of a set of parameters composes a distribution NT, T for representing a distribution of a parameter value of the relevant parameter in a control group and in a case group respectively. Typically the set of parameters may include 5 to 20 parameters. However, also a lower or higher number of parameters may be involved. The distributions NT, T, may be obtained with distribution composing module 50. Alternatively respective distribution composing modules may be provided to determine each of the distributions NT, T.

A first distribution evaluation module 61 determines the characteristics of the distribution NT, such as specific percentile values (P_{5,NT}), (P_{10,NT}), (P_{90,NT}), (P_{95,NT}). A second distribution evaluation module 62 determines corresponding characteristics of the distribution T, such as specific percentile values (P_{5,T}), (P_{10,T}), (P_{90,T}), (P_{95,7}). Alternatively a single distribution evaluation module may be provided to determine the characteristics of both distributions NT, T for the biomarker.

One or more comparison modules are provided that compare the characteristics of the distributions NT, T. In this case a first comparison module 71 is provided to compare distribution characteristics related to the left tail of the distributions NT, T and a second comparison module 71 is provided to compare distribution characteristics related to the right tail of the distributions NT, T. Based on the received distribution characteristics these comparison modules respectively determine which of the two distributions define the value ranges for the partial prediction value of the left tail and the right tail respectively.

As indicated above, the set of parameters typically includes a plurality of parameters. As another example of a parameter it is now illustrated how the various value ranges are obtained for the parameter PAPPAMoM.

In this example, for a first predetermined percentile lower than 50, in this case the 10^{th} percentile (P_{10,NT}) for the first one (NT) of these distributions is 0.42, which is clearly higher that that of the second distribution.

Hence the distribution NT is selected by comparison module 71 to define the value ranges.

Now for this selected distribution NT the value of a second predetermined percentile, here equal to the first predetermined percentile, in this case the 10^{th} percentile is determined, which is 0.42.

Based on the value for the second predetermined percentile of the selected distribution NT a first value range is defined by a first partial prediction value to range assignment module 81 having said parameter value as an upper bound and a second value range having said parameter value (P_{10,NT}) as a lower bound. As in this case the selected distribution is the first distribution NT a partial prediction value is assigned by the first assignment module 81 to the value ranges, wherein a partial prediction value assigned to the first value range contributes more to the combined prediction value PV_{fin} predicting the condition trisomy than a partial prediction value assigned to the second value range. The partial prediction value may be assigned by assignment module. In this case parameter values higher than 0.42 (P_{10,NT}) do not contribute to the combined prediction value, e.g. they are assigned a partial prediction value 0 by this module 81.

In particular, as indicated in Table 1 above, the first value range ValRange1 is subdivided into the following subranges with corresponding partial prediction values.

A range 0-0.19 (P_{1,NT}) with partial prediction value 7
A range 0.19 (P_{1,NT})-0.33 (P_{5,NT}) with partial prediction value 2
A range 0.33 (P_{5,NT})-0.42 (P_{10,NT}) with partial predication value 1.

As indicated above, for each of the distributions (T, NT) the first and the second distribution evaluation module 61, 62 determine a respective parameter value for a third predetermined percentile higher than 50.

The distribution T, which has the lowest value for the third predetermined percentile is selected by the second comparison module 72 to define the value ranges. Hence, based on the value for the second predetermined percentile of the selected distribution T, a third and a fourth value range are defined by the second partial prediction value to range assignment module 82. Therewith a boundary between a third value range and a fourth value range can be defined by a fourth predetermined percentile of this distribution, wherein the third value range has the fourth predetermined percentile (P_{90,T}) of the selected distribution T as its upper bound and wherein the fourth value range has this fourth predetermined percentile (P_{90,T}) as its lower bound. In this case the fourth predetermined percentile is equal to the third predetermined percentile (P_{90,T}) for that distribution T, which has the value 0.88.

As the selected distribution T in this case is the second distribution pertaining to the group having the diagnosis trisomy, a partial prediction value is assigned by the second assignment module 82 to the value ranges, such that a partial prediction value assigned to the fourth value range contributes more to said combined prediction value predicting the absence of said condition than a partial prediction value assigned to the third value range if the selected distribution.

Here, the fourth value range is subdivided into the following subranges with corresponding partial prediction values.

a subrange in between P_{90,T} and P_{95,T}, with partial prediction value −1
a subrange in between P_{95,T} and P_{99,T}, with partial prediction value −2
a subrange in between P_{99,T} and P_{100,T}, with partial prediction value −7

As a result the design apparatus AI prepares a complete set of value ranges, and partial prediction value data (VR_{1,BM1},PP₁; V_{R2,BM1},PP₂, VR_{3,BM1},PP₃, VR_{4,BM1},PP₄), (VR_{1,BM2},PP₁; . . . )). The set specifies for each parameter BM1, BM2, . . . , BMn a set of value ranges and associated partial prediction values, e.g. (VR_{1,BM1},PP₁; VR_{2,BM1},PP₂, VR_{3,BM1},PP₃, VR_{4,BM1},PP₄) and provides this set of data to the indication apparatus AII.

The design method and design apparatus in this way provide a normalized set of evaluation data allows the indication apparatus AII to process individual diagnostic data in a uniform manner, as described below.

As shown in FIG. 4, the indication apparatus includes a parameter value issuing module 10 to issue respective individual values (VBM₁, VBM₂, . . . , VBM_{N}) for a set of indicative parameters (BM₁, BM₂, . . . , BM_{N}) indicative for a particular condition with an individual. The set of indicative parameters may for example include the parameters referred to in Tables 1,2 above, and the values VBM₁, VBM₂, . . . , VBM_{N} are the values for these parameters determined for that individual. The parameter value issuing module 10 may include one or more units to be determined with the individual, for example by measuring the amount of said biomarker in a urine or a serum sample of an individual. Alternatively, the parameter values may have been determined at an earlier point in time and may be issued by a parameter value issuing module 10 in the form of a reading unit for reading a value of a parameter from a storage unit.

The value range and associated partial prediction values can subsequently be used by an evaluation module 20. The evaluation module receives representative input data from an individual. By way of example it is shown how the evaluation module 20 includes a first evaluation unit 21 that evaluates the value V_{BM1} of the first biomarker BM₁ (e.g. PAPPAMoM) with respect to the evaluation data (VR_{1,BM1},PP₁; VR_{2,BM1},PP₂) obtained by the apparatus AI in FIG. 4 or the method of FIG. 1. Similarly the evaluation module 20 includes a second evaluation unit 22 that evaluates the value V_{BM1} of this biomarker BM₁ with respect to the evaluation data (VR_{3,BM1},PP₃; VR_{4,BM1},PP₄). Typically the ranges do not overlap, so that at most one of the evaluation units 21 issues a partial prediction value (PV_{BMIL}, PV_{BMIR}) that can contribute to the combined prediction value. For example in the case of the parameter PAPPAMoM, the first evaluation unit 21 issues a value PV_{BMIR} that contributes to a combined prediction value indicative of trisomy if the individual has a parameter value less than 0.42 for this parameter and the second evaluation unit 22 issues a value PV_{BMIR} that contributes to a combined prediction value indicative that trisomy is unlikely if the individual has a parameter value higher than 0.88 for this parameter.

Similarly a partial prediction value can be determined for other parameter values (VBM₂, . . . , VBM_{N}). A combination module 30 then determines the combined prediction value based on the partial prediction values issued by all evaluation modules.

As in the indication apparatus AII according to the present invention each parameter value is assigned a normalized partial prediction value in accordance with the evaluation data provided by the design method of FIG. 1 or the design apparatus AI in the upper portion of FIG. 4, the resulting normalized partial prediction values obtained can be processed in a uniform manner. Per case, these normalized values can be summed by the combination module 30 therewith obtaining the combined prediction value that can be considered as an index. If desired an additional weighting may be applied to the partial prediction values before combining. For example if for the relative contribution of a biomarker is low (as defined by its contribution to the AUCᵣₑₐₗ, (see Table 4), it may be assigned a weight lower than that of the other biomarkers.

As indicated above, each biomarker used by the apparatus AII has a first distribution of values in a control group which according to a Golden Standard does not have said condition and a second distribution of values in a group of entities for which said condition is determined according to said Golden Standard. Each of said distributions has a respective parameter value (P_{10,NT}, P_{10,T}, P_{5,NT}, P_{5,T}; P_{90,NT}, P_{90,T}; P_{95,NT}, P_{95,T}) for a first percentile (e.g. 10) lower than 50, a second percentile (e.g. 5) lower than 50, a third percentile (e.g. 90) higher than 50, and a fourth percentile (95) higher than 50. A first selected distribution is denoted as the distribution selected from the first and the second distribution that has a highest parameter value for the first percentile. A second selected distribution is denoted as the distribution selected from the first and the second distribution that has a lowest parameter value for the third percentile,

Upon inspection of the apparatus AII of FIG. 4, it will become apparent that the following relationships exist between the evaluation criteria for a parameter on the one hand and the first and the second distribution of the parameter in the control group and the case group respectively:

a) The first value range VR_{1,BM1} has the parameter value of the first selected distribution for the second percentile as an upper bound and a second value range VR_{2,BM1} has said parameter value as a lower bound.
b1) if the first selected distribution is the first distribution then a partial prediction value PP₁ assigned to the first value range VR_{1,BM1} contributes more to said combined prediction value PV_{fin} predicting said condition than a partial prediction value PP₂ assigned to the second value range VR_{2,BM1}.
b2) if the selected distribution is the second distribution then a partial prediction value PP₁ assigned to the first value range VR_{1,BM1} contributes more to said combined prediction value PV_{fin} predicting the absence of said condition than a partial prediction value assigned to the second value range.
c) The third value range VR_{3,BM1} has the parameter value of the second selected distribution as an upper bound and the fourth value range VR_{4,BM1} has said parameter value as a lower bound,
d1) If the second selected distribution is the first distribution then a partial prediction value PP₄ assigned to the fourth value range VR_{4,BM1} contributes more to said combined prediction value PV_{fin} predicting said condition than a partial prediction value PP₃ assigned to the third value range VR_{3,BM1}.
d2) If the second selected distribution is the second distribution then a partial prediction value PP₄ assigned to the fourth value range VR_{4,BM1} contributes more to said combined prediction value PV_{fin} predicting the absence of said condition than a partial prediction value PP₃ assigned to the third value range VR_{3,BM1}.

The combined prediction value PV_{fin} as obtained above, is indicative for the likelihood of a condition with an individual. For example, the combined prediction value computed from the partial prediction values as described with reference to tables 1,2 indicates the risk that a mother gives birth to a child having trisomy and is herein denoted as Trisomy Index (TI). As another example the combined prediction value computed from the partial prediction values obtained for biomarkers of Annex 2 indicates the likelihood that the individual suffers from a depression, and the combined prediction value so obtained is defined herein as Bio-Depression-Score, denoted herein also as BDS.

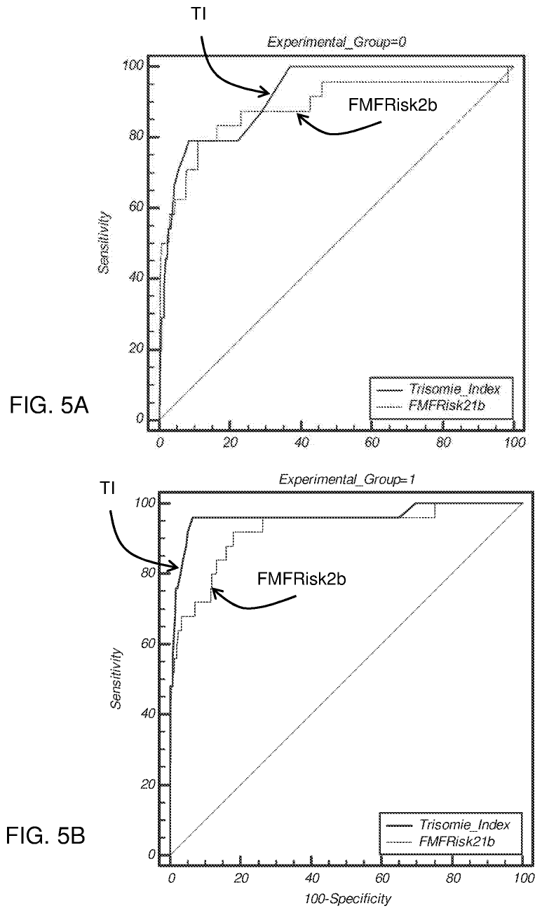

In FIG. 5A, the performance of the combined prediction values so obtained for the individuals, in this example named Trisomy Index (TI), is illustrated graphically as the area under the curve defined by the sensitivity as a function of the inverse specificity (100-specificity) and is compared with a commercially available indicator FMFRisk2b. The selected group is the training group.

Using the AUC analysis of the Trisomy Index, the optimal Cut-off value for the Trisomy Index appears ≥2 which leads to a sensitivity of 96.0% and a specificity of 90.1%. The FMFRisk2b was set at 1:200 and leads to a sensitivity and specificity of 72.0% and 88.5% respectively (Table 5).

Using this cut-off value, a risk-assessment for the actual occurrence of a trisomy can be performed for the trisomy-index and the FMFRisk21b (reference). The results are shown in Table 5.

TABLE 5
Comparison trisomy Index (TI) and FMFRisk2b relative to a child with trisomy diagnosed after birth
validation set (n = 1642)

| | | Trisomy Index | | FMFRisk21b | | | | Trisomy | |
|---|---|---|---|---|---|---|---|---|---|
| | | TI ≥ +2 | TI < +2 | Pr > 1:200 | Pr < 1:200 | total | | Index | FMFRisk21b |
| Trisomy | Yes | 24 | 1 | 18 | 7 | 25 | sensitivity | 96.0% | 72.0% |
| | No | 160 | 1457 | 186 | 1431 | 1617 | specificity | 90.1% | 88.5% |
| | PPV | 13.0% | | 8.8% | | | | | |
| | NPV | | 99.93% | | 99.51% | | | | |

| | | | | |
|---|---|---|---|---|
| Pr on trisomy 1: | 8 | 1458 | 11 | 205 |

A Negative result (TI<2) reveals a chance of 1:1458 for the occurrence of a trisomy, whereas a Positive result (TI≥2) reveals a chance of 1:8. The equivalent results for the FMFRisk2b reference are 1:205 and 1:11.

To assess a difference in frequency between Positive result and Negative result, a chi-square test is applied. No differences in observed frequencies are found for a Pos result: 24/136 vs 18/186, p=0.24). For a Negative result the frequency of a positive result by the TI-method (1/1457) is lower than in the reference FMFRisk21b method (7/1431). However, this difference is statistically not significant (p=0.07).

FIG. 5B shows the sensitivity—inverse specificity curves for the validation group. An AUC comparison performed on these curves for the TI-index and FMFRisk21b index reveals a clear difference in that AUC-TI=0.961 and AUC-FMFRisk21b=0.923. This difference is statistically significant: p=0.018.

Thus, the newly developed method allows for a reliable analysis and identification of multiple parameters that contribute to the diagnosis of a disease. Whereas in the example presented above the partial prediction value is assigned as a stepwise function of the parameter value, also other assignments of the prediction values to the respective value ranges are possible. For example the first prediction value to range assignment value module 21 may assign a partial prediction value that gradually decreases as a function of the parameter value for the parameter PappaMom. For example the assigned prediction value may be a function that monotonically decreases from 7 for a value of 0 for said parameter to 0 for a value of 0.5 of said parameter.

### Example 2: Major Depressive Disorder (MDD)

As another example the design method according to the present invention was applied to select proper biomarkers and to design evaluation criteria for computing a combined prediction value for the condition of a Major Depressive Disorder (MDD). The latter is a heterogeneous disorder with a considerable symptomatic overlap with other psychiatric and somatic disorders. As a result, diagnosis may be complicated, particularly for the non-psychiatrist physician. Accordingly, there is a need for a practical clinical test to assist in the diagnosis of MDD by testing a small set of serum and/or urine biomarkers. To this end, urine and serum samples of 51 MDD patients as well as 51 age-, sex-, and ethnicity-matched controls for levels of 40 potential MDD biomarkers (21 serum biomarkers and 19 urine biomarkers) were analyzed. The selection procedure as described with reference to FIG. 3 was employed to select biomarkers on the basis of differences in variation and distribution between groups. Depression probability scores (the “bio depression score”) were calculated by combining the outcomes of the selected biomarkers, and calculated for clinical discrimination of depressed from euthymic subjects. Based on this algorithm, a combination of 11 urine biomarkers and 6 serum biomarkers was identified with an area under the curve (AUC) of 0.889 in the AUC analysis. Selection of either urine biomarkers or serum biomarkers resulted in somewhat lower AUC values, amounting to 0.859 and 0.755, respectively. A phenotype permutation analysis showed a significant discrimination between MDD and euthymic (control) subjects for biomarkers in urine (P<0.001) and for the biomarkers in serum and urine combined (P<0.001), but not for serum biomarkers only (P=0.20). An internal cross-validation confirmed the predictive value of this set of biomarkers.

The application of the inventive design method to design a practical clinical test to assist in the diagnosis of MDD is now discussed in more detail. As indicated above several biomarkers have been suggested to be indicative for MDD. Examples thereof include cytokines (e.g. TNFα, IL-1ß), neurotrophic factors (e.g. BDNF, VEGF), and hormones (e.g. cortisol). However, none of these biomarkers fulfill the sensitivity and specificity criteria when used individually. This may be in part due to the complicated underlying pathophysiology of MDD. An increasing body of evidence indicates that the underlying neurobiology of MDD likely involves a complex interplay of genetic factors, dysregulation of the hypothalamic-pituitary-adrenal (HPA) axis and other endocrine parameters, dysfunctions in the immune system and monoaminergic systems. Accordingly, single genetic, endocrinological, neurotransmitter-related or hormonal abnormalities are unlikely to discriminate patients with severe mood disorders from healthy people or patients with other psychiatric disorders. Combining a number of biomarkers reflecting the divergent dysfunctions in MDD might be a more fruitful approach [3].

A major problem in biomarker-based diagnostics is the fact that biomarker values are not normally distributed and distributions may be different in patients and healthy controls. When the distribution in patients and healthy controls differs in aspects other than the mean or median, difficulties arise for parametric and non-parametric testing. Examples in which regular parametric or non-parametric testing fails, include a ceiling effect in one of the groups or differences in variance between groups not accompanied by differences in average. Variance information gets lost, when not taking into account that each biomarker obeys different variance rules between cases and controls.

The present invention addresses these pitfalls. The design method selects those biomarkers that “behave” differently between cases and controls while not necessarily displaying a difference in average between both groups. It distinguishes the distributional tail behavior between cases and healthy controls. Using the procedure as described with reference to FIG. 3 the best performing biomarkers were selected, and validity of this subset was tested. The indication method for example when performed with an indication apparatus AII as described with reference to FIG. 4 calculates a depression probability score for clinical discrimination between depressed and euthymic subjects. The indication method and/or indication apparatus provides for each subject a combined prediction value, arbitrarily called Bio-Depression-Score, denoted herein as BDS.

In preparation for the design method, MDD patients were recruited in collaboration with general practitioners, psychiatric clinics and through advertisements in local and national newspapers. Inclusion criteria included: age 18-65, fulfilled DSM-IV criteria for unipolar MDD, a HAM-D score higher than 10 and informed consent. Exclusion criteria included pregnancy, presence of another primary psychiatric disorder, alcohol or substance use disorders, inflammatory or systemic diseases, metabolic disorders or other disorders that might affect mood. Patients with (n=30) and without (n=21) anti-depressant medication were included. Healthy controls (HC) were recruited via general practitioners and advertisements in local and national newspapers. Healthy controls had to be free of any major axis I diagnosis and were matched for gender, age and ethnicity.

The 17-item Hamilton Depression Rating Scale (HAM-D) was used to assess symptoms of depression. In addition, a Mini International Neuropsychiatric Interview (MINI) was conducted. A researcher trained in the use of these questionnaires executed all questionnaires, and an experienced psychiatrist performed the final MDD diagnosis.

Participants were asked to deliver 50 ml of blood through venipuncture as well as 50 ml of first morning urine. Blood was collected in serum separation tubes, allowed to clot and centrifuged at 3000×g for 10 minutes. Serum supernatant was divided into aliquots and stored at −80° C. Urine samples were centrifuged for 10 minutes at 1000×g to precipitate any particles and cells; the supernatant was collected, divided into aliquots and stored at −80° C.

A primary selection of biomarkers to be tested in serum and in first morning urine was based on a thorough literature search in combination with a pilot study in 24 participants (12 MDD patients and their sex, age and ethnic matched healthy controls). The biomarkers included in this pilot cohort and their selection for the follow-up cohort is provided in Annex 2. The selected biomarkers were subsequently tested in a cohort of 51 MDD patients and 51 matched healthy controls. The results of this cohort were subsequently used for the design of an algorithm leading to the diagnostic score (BDS, see below) and statistical validation by permutation analysis. After elimination of non-contributing biomarkers the predictive value of the diagnostics score was investigated by 5-fold cross validation.

### Descriptive Statistics.

Descriptive statistics were calculated for the demographic parameters to describe the population. Numerical variables were summarized with means and standard deviations, while categorical variables were summarized with counts and percentages. Table 6 shows the demographic characteristics of the subjects that were included in the 102 participant cohort. Subjects were matched for sex, age and ethnicity. Control subjects had an average HAM-D17 score of 2.7 (range 2-8), while MDD subjects had an average HAM-D17 score of 23.7 (range 11-43; p<0.0001).

TABLE 6
Demographic characteristics.

| | | All | Healthy | | |
|---|---|---|---|---|---|
| | | participants | Controls | MDD | Statistics |
| Sex | Male | 44 | 22 | 22 | Chi-square |
| | Female | 58 | 29 | 29 | (2 × 2): |
| | | | | | P = 0.84 |
| Age (Yr) | Mean | 46.6 | 47.1 | 46.2 | Mann- |
| | SD | 11.35 | 11.0 | 11.5 | Whitney-U: |
| | | | | | P = 0.81 |
| Ethnicity | Dutch | 88 | 47 | 41 | Chi-square: |
| | Indonesian | 4 | 2 | 2 | (7 × 2) |
| | Surinam | 5 | 2 | 3 | P = 0.19, |
| | Maroc | 2 | 0 | 2 | for non-Dutch |
| | Assyric | 1 | 0 | 1 | (2 × 2): |
| | Brazil | 1 | 0 | 1 | P = 0.15 |
| | Iraq | 1 | 0 | 1 | |
| HAMD17 | mean | N.A. | 2.7 | 23.7 | p < 0.0001 |
| | SD | N.A. | 1.1 | 8.4 | |

ELISA kits, as specified in more detail in Annex 1, were used to obtain the biomarker levels for the participants in the control group and the case group. All procedures were performed according to the manufacturer's instructions making use of an ELISA plate washer PW40 (Sanofi Pasteur). Read-outs of the Microtiter plate were digitally saved. Data were analyzed by making use of standard curves of OD values obtained by the Microtiterplate reader (Multiscan EF type 35, ThermoScientific) against (log transformed) concentrations as provided by the individual manufacturers of the kits. Individually measured patient sample values were obtained by linear interpolation of the sample OD value and the OD values of the standard. From each serum and urine sample creatinine levels were assessed and urine biomarker levels were corrected for the creatinine content. Patients and controls were only included with serum creatinine concentration within the normal range (excluding renal dysfunction). Due to insufficient amount of serum or identification errors, certain ELISAs were excluded in a minority of participants: 2 biomarkers were tested in all participants, 11 biomarkers in all controls and 50 MDD patients, 5 biomarkers in all controls and 49 MDD patients, and 2 biomarkers in 50 controls and 49 MDD patients. The results in serum are expressed as a concentration of the biomarker. The results in urine are expressed as the ratio of biomarker to creatinine by dividing the biomarker concentration by that of creatinine. As a control for normal renal function, creatinine concentration was measured in serum as well and checked to remain within normal value ranges. Only those within the normal serum creatinine range were included. To determine median and variance differences in each biomarker for the MDD and HC group, the Mann-Whitney U test and Levene's test on heterogeneity were applied, respectively. These analyses were all performed with SPSS statistical software, version 23. Table 7 shows the Mann-Whitney U and Levene's test for each biomarker tested in serum and in urine. The Mann-Whitney U test, a test for differences in medians, found only a significant difference for Aldosterone in urine and no differences in serum. The Levene's test, a test on differences in variances, found significance for 6 biomarkers, 4 in serum (BDNF, Isoprostane, TNF-R2, Zonulin) and 2 in urine (LTB4 and Thromboxane). Thus, the traditional (non-parametric) Mann-Whitney U test found only a small number of biomarkers that showed significant differences between MDD and healthy controls and thus limited information would be present to discriminate between these two groups, let alone to predict disease. The test on variability however indicates that there are possible differences in variance that may contribute to the BDS.

TABLE 7
Mann-Whitney U test and Levene's test
results of the 2 × 51 cohort.

| | P-value | | | |
|---|---|---|---|---|
| | Serum | | Urine | |
| | Mann- | | Mann- | |
| | Whitney | Levene's | Whitney | Levene's |
| Biomarkers | U test | test | U test | test |
| Adiponectin | — | — | 0.199 | 0.435 |
| Aldosterone | 0.435 | 0.501 | 0.019* | 0.720 |
| BDNF | 0.595 | 0.032* | — | — |
| Calprotectin | 0.147 | 0.170 | 0.420 | 0.640 |
| cAMP | 0.795 | 0.137 | 0.708 | 0.197 |
| cGMP | — | — | 0.799 | 0.451 |
| Cortisol | 0.169 | 0.094 | 0.748 | 0.052 |
| EGF | 0.231 | 0.455 | 0.392 | 0.720 |
| Endothelin | 0.261 | 0.098 | 0.512 | 0.208 |
| HVEM | 0.377 | 0.759 | 0.357 | 0.178 |
| Isoprostane | 0.658 | 0.008* | 0.284 | 0.984 |
| Leptin | 0.669 | 0.223 | 0.630 | 0.809 |
| Lipocalin | 0.413 | 0.550 | 0.239 | 0.256 |
| LTB4 | 0.651 | 0.711 | 0.928 | 0.016* |
| Midkine | 0.312 | 0.992 | 0.088 | 0.356 |
| Nitrotyrosin | 0.408 | 0.015 | — | — |
| NPY | 0.558 | 0.782 | 0.939 | 0.870 |
| Pregnenolone | — | — | 0.846 | 0.392 |
| Substance P | 0.209 | 0.953 | 0.139 | 0.132 |
| Telomerase | 0.183 | 0.301 | — | — |
| Thromboxane B2 | 0.081 | 0.666 | 0.254 | 0.011* |
| TNF R2 | 0.548 | 0.001* | 0.531 | 0.157 |
| Vitamin D | 0.962 | 0.127 | — | — |
| Zonulin | 0.946 | 0.015* | — | — |

*Biomarkers with a p < 0.05.

### The Design Method.

The design method according to the present invention as presented for example in FIG. 1 is now applied to the results obtained for the control group (HC) and the case group (MDD).

In the first step (corresponding to steps S24L and S24R of FIG. 1), for each of the biomarkers separately, it is determined which of the first distribution (the distribution of a biomarker for the control group HC) and the second distribution (the distribution of a biomarker for the case group MDD) dominates the left tail, and which one the right tail. Therein a distribution for a biomarker is considered to be the dominant one if its 10^{th} percentile has value is lower than that of the other distribution. I.e. for the left tail. MDD dominates if the 10^{th} percentile of the MDD group lies left of the 10^{th} percentile of the HC group. HC dominates if the order of these 10^{th} percentiles is opposite. The non-dominant one of the distributions is then defined as the selected distribution. If the MDD-group dominates, the 1^{st} (P1), 5^{th} (P5) and 10^{th} (P10) percentiles of the HC-distribution are used as cut-offs to form scores in the BDS and vice versa. For the right tail, MDD dominates if the 90^{th} percentile of the MDD group lies to the right of the 90^{th} percentile of the HC group. HC dominates when this order is opposite. If the MDD group dominates, the 90^{th} (P90), 95^{th} (P95) and 99^{th} (P99) percentiles of the HC-distribution are used as cut-offs to form scores in the BDS, and vice versa.

Based on the above cut-offs, the distribution of each biomarker is divided into the following 7 parameter value ranges: values≤P1; P1<values≤P5; P5<values ≤P10; P10<values<P90; P90≤values<P95; P95≤values<P99; values≥P99. This corresponds to steps S25L, S25R, S26L, S26R in FIG. 1.

As in step S27L, S27R of FIG. 1, each biomarker value is transformed into a score (partial prediction value). Within the segment P10 to P90 the score will become zero (no contribution to disease prediction). In a MDD dominant tail (HC is the selected distribution) the scores are assigned positive (+1, +2, +3) values, with higher numbers for segments further away in the tails. In a HC dominant tail (MDD is the selected distribution) the score is assigned negative (−1, −2, −3) scores, with lower values for segments further away in the tail.

In the present example, an additional selection corresponding to steps S28L, S28R in FIG. 1 is made as follows. To include a tail for a biomarker, it is required that the tail of the dominant group should extend substantially beyond the non-dominant tail. For instance, the probability mass of the left (or right) tail of the dominant group is required to be at least 20% at the cut-off P10 (P90) of the non-dominant group. In addition, it is required that the probability mass of the dominant group at the left (right) of the cut-off P5 (P95) is more than 15%. If one biomarker tail does not reach these two pre-set tail criteria, the BDS score for participants with biomarker values in these tails are set to zero. If both tails of a biomarker fail to satisfy these tail criteria, the biomarker does not contribute to disease prediction. The criteria in the procedure specified above are summarized in Table 8.

TABLE 8
Summary of assigned normalizing values in all options

| Dominance in tail | | | Partial prediction values in the different segments | | | | | | |
|---|---|---|---|---|---|---|---|---|---|
| | Left | Right | ≤P_{1, s} | >P_{1, S}-≤P_{5, S} | >P_{5, S}-≤P_{10, S} | >P_{10, S}-<P_{90, S} | ≥P_{90, S}-<P_{95, S} | ≥P_{95, S}-<P_{99, S} | ≥P_{99, S} |
| Both | HC | MDD | −3 | −2 | −1 | 0 | 1 | 2 | 3 |
| Tails | HC | HC | −3 | −2 | −1 | 0 | −1 | −2 | −3 |
| | MDD | HC | 3 | 2 | 1 | 0 | −1 | −2 | −3 |
| | MDD | MDD | 3 | 2 | 1 | 0 | 1 | 2 | 3 |
| One | HC | — | −3 | −2 | −1 | 0 | 0 | 0 | 0 |
| Tail | — | HC | 0 | 0 | 0 | 0 | −1 | −2 | −3 |
| | MDD | — | 3 | 2 | 1 | 0 | 0 | 0 | 0 |
| | — | MDD | 0 | 0 | 0 | 0 | 1 | 2 | 3 |
| None | — | — | 0 | 0 | 0 | 0 | 0 | 0 | 0 |

Accordingly, each participant obtains per biomarker a score ranging from −3 to +3. The BDS (combined prediction value) for a participant is the sum of the scores (partial prediction values) over the biomarkers. Thus the BDS is the cumulative information from all incorporated biomarkers towards the presence or absence of MDD. A positive score indicates a preference for the disease, while a negative score indicates a preference for healthy. A score of zero implies there is no preference. The higher the score the more likely the disease is present and the lower the score the more likely the disease is not present.

Based on the BDS of the participants and the disease classification an area-under-the-curve (AUCReal) can be calculated. MedCalc Statistical Software version 16.8 (or later) is used for comparison differences of ROC curves between the groups serum only, urine only and serum plus urine. The larger the AUCReal, the better the BDS discriminates between healthy and disease and the more it contains real information for the diagnosis of MDD. The AUCReal is used to determine the optimal two pre-set criteria on the tail dominance mention in step S28L, S28R above.

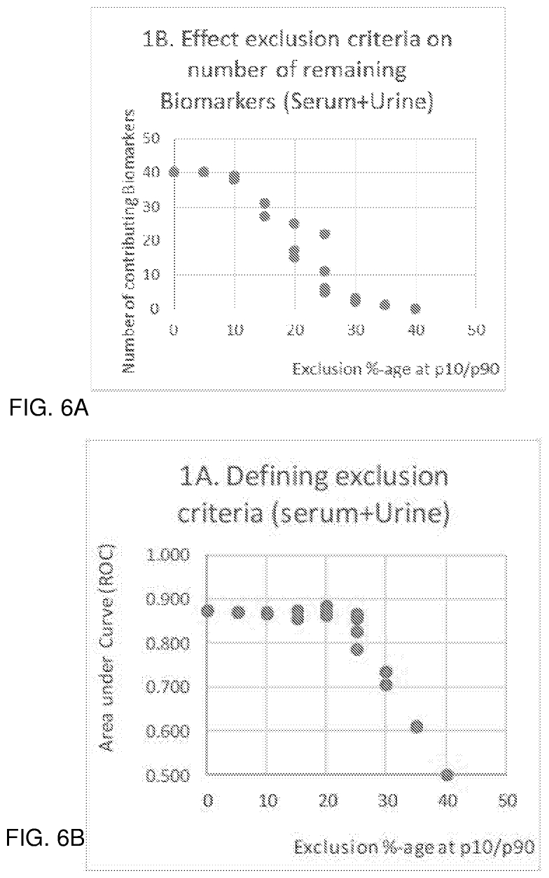

The optimal criteria for exclusion of non-performing (tails of) this set of biomarkers is investigated by varying the criteria for P10/P90 and P5/P95 from 0% to 40% and checking the effect on the AUC in Group 3 (Serum+Urine). FIGS. 6A and 6B show the results. Therein FIG. 6A shows the number of remaining biomarkers that fulfill the additional requirement, and FIG. 6B shows the AUC that is obtained with this remaining set of biomarkers. In the absence of any requirements apart from the requirements imposed by the selected distribution, all 40 biomarkers are included and an AUC of 0.875 is obtained. With the requirement of a probability mass of the dominant group of at least 40% for the P10 and P90 all biomarkers become excluded as a result of which the AUC becomes 0.500. At a mass at 20% for P10 and P90 and at 15% for the P5 and P95 of the dominant group, the AUC is maximal (AUCReal=0.889). These conditions exclude 23 biomarkers thus leaving 17 (6 in Serum and 11 in Urine). At higher %-ages the AUC drops sharply. Thus, exclusion P10/90 at 20% and P5/95 at 15% is the condition generating the optimal result, which is chosen for further analysis.

To determine the significance of the discrimination of BDS, ‘phenotype randomization’ was applied. Phenotype randomization or permutation consists of randomly redistributing the classification of MDD and HC over the original biomarker data: thus the biomarker data per participant are kept unchanged but the labels MDD and HC are permuted at random (as in step S131 of FIG. 3). Then, a BDSRandom is generated identically as described in S132 of FIG. 3 and in step S133, the ROC analysis on BDSRandom results in an AUCRandom. Repeating (S130) this phenotype randomization 10000 times generates (S140) an AUCRandom frequency distribution of which many should be non-discriminative with respect to the disease classification since the relation between biomarker and disease is destroyed by many of the permutations. The p-value is now defined by the fraction of all AUCRandom permutations that are larger or beyond the AUCReal. A p-value below 0.05 indicates a significant discrimination of the BDS and the null hypothesis that the BDS does not discriminate should be rejected.

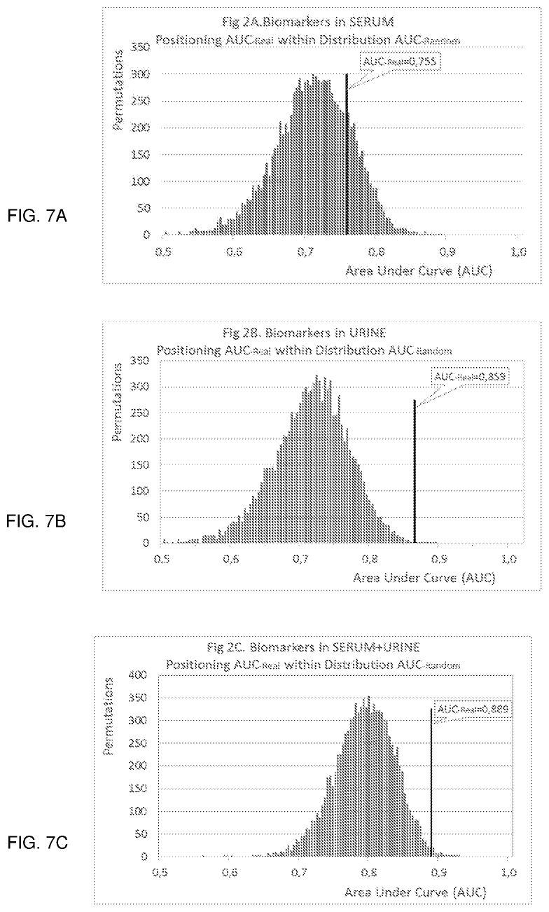

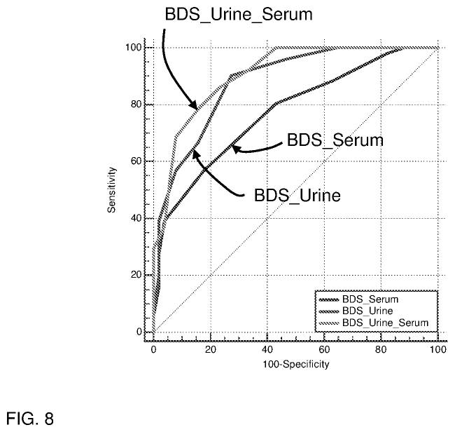

The BDS was calculated for each of the 3 groups. Group 1 uses the information of the 21 biomarker levels in serum only. Group 2 uses on the information of the 19 biomarkers in urine only. Group 3 uses all 40 biomarkers. The BDS was calculated according to the algorithm described in Step 1 of the Methods section. Non-performing biomarker (tails) were identified (Step 1.4) and details are described in supplementary information S5. A total of 23 biomarkers were excluded leaving 17 biomarkers (6 in serum, 11 in urine) to form the BDS. The included serum biomarkers are TNF-R2, Cortisol, Calprotectin, Thromboxane, Endothelin and Leptin. The included urine biomarkers are cGMP, Calprotectin, Leptin, LTB4, Cortisol, Thromboxane, Isoprostane, Aldosterone, HVEM, Midkine and Substance P. The ROC curves showing the results of the included biomarkers for all subjects are visualized in FIG. 8. The AUC values derived from these curves are: 0.755 for the 6 serum biomarkers, 0.859 for the 11 urine biomarkers and 0, 889 for the 17 combined serum+urine biomarkers. Comparison of ROC curves: ‘Serum’ versus ‘Urine’: P=0.04; ‘Serum’ versus ‘Serum plus Urine’: P=0.0003; ‘Urine’ versus ‘Serum plus Urine’: p=0.21.). The frequency distributions of the AUC_{Random}, with the AUC_{Real} are visualized for serum, urine, and urine and serum respectively in FIGS. 7A-7C.

To determine the predictive value of the BDS a (five-fold) cross-validation was performed. The validation was done on the biomarkers that were included by the algorithm on the whole data. The 102 participant cohort was randomly divided into five parts (20, 20, 20, 20 and 22 participants) such that each part contains an equal number of MDDs and HCs. No attempt was made to match age, sex or ethnicity. Five separate sets were constructed as indicated in Table 9 below, such that each contains 4 of the 5 parts for training and 1 of the 5 parts for validation and prediction. For each set separately, the participants in the ‘training part’ were used to determine in step S25L of FIG. 1 the cut-offs for the percentiles P1, P5, P10, P90, P95 and P99, using the obtained predominance of biomarkers in the full data. Given these cut-offs, a BDS can then be calculated for participants in the validation part together with the ROC-curve for classification of the disease (MedCalc Statistical Software version 16.8 including binomial exact confidence interval for the AUC). To repeat this for each training set we obtain five sets of predicted AUC's (for serum only, urine only, and serum and urine simultaneously). These results provide the predictive value of the BDS to classify MDDs from HCs as indicated.

TABLE 9
Experimental set-up cross-validation.
Table xx: Internal Cross-Validation:
experimental set up: samples in 5 parts

| Healthy | | | | | |
|---|---|---|---|---|---|
| (51) and | | | | | |
| MDD (51) | 1-20% | 21-40% | 41-60% | 61-80% | 81-100% |
| Set 1 | Training | Training | Training | Training | Validation |
| | (2 × 10) | (2 × 10) | (2 × 10) | (2 × 10) | (2 × 11) |
| Set 2 | Training | Training | Training | Validation | Training |
| | (2 × 10) | (2 × 10) | (2 × 10) | (2 × 10) | (2 × 11) |
| Set 3 | Training | Training | Validation | Training | Training |
| | (2 × 10) | (2 × 10) | (2 × 10) | (2 × 10) | (2 × 11) |
| Set 4 | Training | Validation | Training | Training | Training |
| | (2 × 10) | (2 × 10) | (2 × 10) | (2 × 10) | (2 × 11) |
| Set 5 | Validation | Training | Training | Training | Training |
| | (2 × 10) | (2 × 10) | (2 × 10) | (2 × 10) | (2 × 11) |

Per training-validation set, the percentile cut-off values in the training subset are determined and applied to the validation subset, leading to predicted AUC-values as presented in Table 10 below. The mean AUC of the ROC curves is lowest for the serum biomarkers, followed by the urine biomarkers and highest in the combined serum and urine biomarkers. Concomitantly, the confidence intervals in AUC values of ROC curves in the Validation parts range from 0.418 to 0.988 for serum biomarkers, from 0.488 to 0.995 for urine biomarkers, and from 0.569-0,995 for serum plus urine biomarkers. The lowest value is found in serum, followed by urine and highest in the combination serum plus urine.

TABLE 10
Results of five-fold cross validation expressed in the AUC's of the Validation
sub sets. V1, V2, V3, V4, V5: the Validation subset in the 1^{st}, 2^{nd}, 3^{rd}, 4^{th} and
5^{th} cross-validation experiment. Therein CI is the Confidence interval

| Serum | 6 BM's | | Urine | 11 BM's | | Serum plus urine | 17 BM's | |
|---|---|---|---|---|---|---|---|---|
| Validation | AUC | 95% CI | validatie | AUC | 95% CI | Validation | AUC | 95% CI |
| V1ₛₑᵣᵤₘ | 0.660 | 0.418-0.853 | V1ᵤᵣᵢₙₑ | 0.860 | 0.633-0.972 | V1ₛₑᵣᵤₘ₊ᵤᵣᵢₙₑ | 0.805 | 0.569-0.945 |
| V2ₛₑᵣᵤₘ | 0.905 | 0.696-0.988 | V2ᵤᵣᵢₙₑ | 0.795 | 0.558-0.940 | V2ₛₑᵣᵤₘ₊ᵤᵣᵢₙₑ | 0.840 | 0.609-0.963 |
| V3ₛₑᵣᵤₘ | 0.907 | 0.677-0.992 | V3ᵤᵣᵢₙₑ | 0.730 | 0.488-0.901 | V3ₛₑᵣᵤₘ₊ᵤᵣᵢₙₑ | 0.870 | 0.645-0.977 |
| V4ₛₑᵣᵤₘ | 0.731 | 0.502-0.895 | V4ᵤᵣᵢₙₑ | 0.845 | 0.615-0.966 | V4ₛₑᵣᵤₘ₊ᵤᵣᵢₙₑ | 0.895 | 0.677-0.986 |
| V5ₛₑᵣᵤₘ | 0.777 | 0.551-0.924 | V5ᵤᵣᵢₙₑ | 0.930 | 0.736-0.995 | V5ₛₑᵣᵤₘ₊ᵤᵣᵢₙₑ | 0.930 | 0.736-0.995 |
| Mean AUC | 0.796 | | | 0.832 | | | 0.868 | |

These predicted results fit with the overall result as presented in FIG. 8

It is noted that the computational resources as used for performing computational steps may be implemented in various ways. For example the computational steps may be performed by a general purpose processor, dedicated hardware, or by a combination of both. One or more of electronic and/or optical computation elements may be employed. Data may be exchanged between components in a wired or wireless manner and may be based on one or more of electrical and/or optical signals.

### Annex 1: ELISA Kits; Manufacturers

ELISA kits were obtained from the following vendors: R&D systems Europe Ltd, Abingdon, United Kingdom (Cortisol, LTB4, Thromboxane, Endothelin-1, Substance P, c-AMP, and c-GMP); Ray Biotech Inc, Norcross, Ga., USA (Leptin, EGF, Lipocalin, adiponectin, TNFalpha receptor 2 and HVEM); Sanbio B, Hycult biotech, Uden, The Netherlands (Calprotectin); Northwest Life Science Specialties, LLC, Vancouver, Wash., USA (Isoprostane-2); Immundiagnostik GmbH, Bensheim, Germany (Zonulin); Cellmid Limited, Perth, Australia (Midkine); Diasource, Leuven, Belgium (Pregnenolone and vitamin D); Peninsula Laboratories, LLC, San Carlos, Calif., USA (NPY); Promega Benelux BV, Leiden, The Netherlands (BDNF). LDN, Germany (Aldosterone); Hycult Biotech, USA (Nitrotyrosin).

Annex 2:
Biomarker selection in the pilot and case/control study

| Biomarkers tested in | |
|---|---|
| 2 × 12 Pilot | 2 × 51 Case/Control |

| Biomarkers | Serum | Urine | Serum | Urine |
|---|---|---|---|---|
| Adiponectin | X | X | NT | X |
| Aldosteron | X | X | X | X |
| Bcl-2 | X | X | NT | NT |
| BDNF | X | NT | X | NT |
| Beclin-1 | X | NT | NT | NT |
| BFGF | X | X | NT | NT |
| BH4 | X | X | NT | NT |
| Biopterin | NT | X | NT | NT |
| Calprotectin | X | X | X | X |
| Calreticulin | X | X | NT | NT |
| CAMK2B | X | X | NT | NT |
| Camp | X | X | X | X |
| CCK | X | X | NT | NT |
| Cgmp | X | X | NT | X |
| Cortisol | NT | X | X | X |
| Digoxine | X | X | NT | NT |
| EGF | X | X | X | X |
| Endothelin | X | X | X | X |
| F2-Isoprostane | X | NT | NT | NT |
| GABA | X | X | NT | NT |
| Galectin-8 | X | X | NT | NT |
| HVEM | X | X | X | X |
| IGF1 | X | X | NT | NT |
| IL-6 | X | X | NT | NT |
| Isoprostane | NT | X | X | X |
| Leptin | X | X | X | X |
| Lipocalin | X | X | X | X |
| LOX1 | X | X | NT | NT |
| LTB4 | X | X | X | X |
| MBP | X | X | NT | NT |
| Midkine | X | X | X | X |
| MMP-1 | X | NT | NT | NT |
| Myeloperoxidase | X | X | NT | NT |
| Neopterin | X | X | NT | X |
| Neuropeptide Y | X | X | X | X |
| NGF | X | X | NT | NT |
| Nitrotyrosine | X | X | X | NT |
| PEDF | X | X | NT | NT |
| PLA2G7-PAF | X | N | NT | NT |
| Pregnenolon | X | X | X | X |
| Prostaglandin E2 | X | X | NT | NT |
| Substance P | X | X | X | X |
| Svegf | X | X | NT | NT |
| Thromboxane B2 | X | X | X | X |
| TNF-R2 | X | X | X | X |
| Vasopressin | X | X | NT | NT |
| VILIP | X | X | NT | NT |
| Vit-D | X | X | X | NT |
| Zonulin | X | X | X | NT |

X: Biomarker tested; NT Biomarker not tested.

1. A computer-implemented design method for providing a definition of an indication method and/or indication apparatus, the design method being configured to define evaluation criteria to be used by the indication method and/or indication apparatus to issue a combined prediction value for the probability of an individual having a condition based on the individual respective parameter values determined for said indicative parameters with said individual and said evaluation criteria, the design method comprising:
a) for each indicative parameter in a set of indicative parameters,
a1) for a first group of entities, according to a Golden Standard not likely to have said condition:
a11) for each individual in the first group obtaining a respective first value for said indicative parameter;
a12) determining a first distribution of the first values obtained for said indicative parameter,
a2) for a second group of entities, according to said Golden Standard likely to have said condition:
a21) for each individual in the second group obtaining a respective second value for said indicative parameter;
a22) determining a second distribution of the second values obtained for said indicative parameter;
a3) for each of said distributions determining a respective indicative parameter value for a first predetermined percentile lower than 50;
a4) selecting from said first and said second distribution the one having the higher parameter value for said first predetermined percentile;
a5) determining for the selected distribution a parameter value associated with at least a second predetermined percentile lower than 50;
a6) defining at least a first value range having said parameter value as an upper bound and a second value range having said parameter value as a lower bound;
a7) assigning a partial prediction value to said value ranges, wherein a partial prediction value assigned to the first value range is a stronger indicator for said condition than a partial prediction value assigned to the second value range if the selected distribution is the first distribution, and wherein a partial prediction value assigned to the first value range is a stronger indicator for the absence of said condition than a partial prediction value assigned to the second value range if the selected distribution is the second distribution,
and/or
a8) for each of said distributions determining a respective parameter value for a third predetermined percentile higher than 50;
a9) selecting from said first and said second distribution the one having the lower parameter value for said third predetermined percentile;
a10) determining for the selected distribution a parameter value associated with at least a fourth predetermined percentile;
a11) defining at least a third value range having said parameter value as an upper bound and a fourth value range having said parameter value as a lower bound;
a12) assigning a partial prediction value to said value ranges, wherein a partial prediction value assigned to the fourth value range is a stronger indicator for said condition than a partial prediction value assigned to the third value range if the selected distribution is the first distribution, and wherein a partial prediction value assigned to the fourth value range is a stronger indicator for the absence of said condition than a partial prediction value assigned to the third value range if the selected distribution is the second distribution;
b) wherein the definition of the value ranges and the assigned partial prediction values determine to which extent values for said indicative parameters determined for a particular individual contribute to the combined prediction value that indicates said condition or the absence thereof with said particular individual, and that is obtained by adding the partial prediction values.

2. The computer-implemented design method according to claim 1, wherein said second predetermined percentile is less than or equal to said first predetermined percentile and/or wherein said fourth predetermined percentile is greater than or equal to said third predetermined percentile.

3. (canceled)

4. The computer-implemented design method according to claim 1, wherein a parameter value in said second value range or in said third value range has no influence on the combined prediction value and/or wherein said second value range and said third value range together form a neutral range, having the parameter value associated with the at least a second predetermined percentile as its lower bound and having the parameter value for the fourth predetermined percentile as its upper bound, wherein a parameter value in said neutral range has no influence on the combined prediction value.

5. (canceled)

6. The computer-implemented design method according to claim 1, wherein said first predetermined percentile is in the range of 5-30, and said third predetermined percentile is in the range of 70-95.

7. The computer-implemented design method according to claim 1, further comprising determining a first probability mass of the non-selected distribution for a value of the selected distribution for a parameter value that corresponds to a fifth predetermined percentile, lower than 50, wherein the fifth predetermined percentile is lower than the first predetermined percentile.

8. (canceled)

9. The computer-implemented design method according to claim 7, comprising setting a difference between the first prediction value and the second prediction value to zero if the first probability mass is less than twice the probability mass for said parameter value corresponding to the fifth predetermined percentile of the selected distribution.

10. The computer-implemented design method according to claim 1, further comprising determining a second probability mass of the non-selected distribution for a value of the selected distribution for a parameter value corresponding to a sixth predetermined percentile, higher than 50, wherein the sixth predetermined percentile is higher than the third predetermined percentile.

11. (canceled)

12. The computer-implemented design method according to claim 10, comprising setting a difference between the third prediction value and the fourth prediction value to zero if the second probability mass is less than twice the probability mass for the parameter value corresponding to the sixth predetermined percentile of the selected distribution.

13. The computer-implemented design method according to claim 7, further comprising:
determining a second probability mass of the non-selected distribution for a value of the selected distribution for a parameter value corresponding to a sixth predetermined percentile, higher than 50, and
comprising assigning a weight 0 to an indicative parameter if the first probability mass is less than twice the probability mass for the parameter value corresponding to the fifth predetermined percentile of the selected distribution and the second probability mass is less than twice the value of the probability mass for the parameter value corresponding to the sixth predetermined percentile of the selected distribution.

14. The computer-implemented design method according to claim 1, wherein a magnitude of the assigned partial prediction values decreases as a stepwise function of a probability mass of the selected distribution for a parameter value within said first and/or said second value range and/or within said third and/or fourth value range.

15. The computer-implemented design method according to claim 1, wherein a magnitude of the assigned partial prediction values decreases as a continuous function of the probability mass of the selected distribution for a parameter value within said first and/or said second value range and/or within said third and/or fourth value range.

16. The computer-implemented design method according to claim 1, comprising selecting the set of indicative parameters from a superset of parameters according to the following procedure:
a) for each parameter in said superset determining a first parameter value distribution for said first group of entities, and determining a second parameter value distribution for said second group of entities;
b) repeating a verification procedure comprising:
b1) for each individual in said first and said second group of entities randomly assigning a vector of parameter values to one of a first and a second auxiliary parameter value distribution, a vector of parameter values being defined as the set of parameter values determined for the superset of parameters with said individual,
b2) For a plurality of entities determining a combined prediction value based on the parameter values determined for said entities while using the first auxiliary parameter value distribution and the second auxiliary parameter value distribution instead of the first parameter value distribution and the second parameter value distribution respectively;
b3) determining a value of a quality measure indicative for the extent to which the combined prediction value obtained with the superset of parameters indicates the presence of the condition according to said Golden Standard;
c) determining a distribution of values obtained for said quality measure obtained by repeating steps b1) to b3) in said verification procedure b);
d) identifying mutually different candidate sets of parameters within said superset of parameters;
e) for each candidate set of said mutually different candidate sets performing the steps of:
e1) determining a value for said quality measure;
e2) determining a statistical significance of the value determined for the quality measure based on the distribution obtained in step c);
f) selecting an optimal set of said mutually different candidate sets of parameters that optimizes a criterion based at least on said statistical significance.

17. The computer-implemented design method according to claim 16, wherein identifying mutually different candidate sets of parameters within said superset of parameters comprises defining for each of said candidate sets respective selection criteria for its parameters, wherein a selection criterion is the magnitude of a probability mass ratio defined as the value of the probability mass of the non-selected distribution divided by the probability mass of the selected distribution for a first parameter value that is determined by the value for the selected distribution at a fifth predetermined percentile lower than 50 or for a second parameter value that is determined by the value for the selected distribution at a sixth predetermined percentile higher than 50, a parameter being an element of a candidate set if its probability mass ratio exceeds a threshold ratio defined for said candidate set at at least said first or said second parameter value, wherein mutually different candidate sets have mutually different values for said threshold ratio.

18. The computer-implemented design method according to claim 16, wherein said quality measure is an area under a curve specifying a relation between the specificity and sensitivity obtained for the set of parameters.

19. A design apparatus for providing a definition of an indication method and/or indication apparatus, the design apparatus being configured to define evaluation criteria to be used by the indication method and/or indication apparatus to issue a combined prediction value for the probability of an individual having a condition based on the individual respective parameter values determined for said indicative parameters with said individual and said evaluation criteria, the design apparatus comprising:
a distribution composing module that for each of a set of parameters composes a first and a second distribution for representing a distribution of a parameter value of the relevant parameter in a control group and in a case group respectively, wherein the control group is a first group of entities, that according to a Golden Standard is not likely to have the condition and wherein the second group of entities, according to said Golden Standard is likely to have said condition;
at least one distribution evaluation module that determines statistical characteristics for said first and said second distribution, said statistical characteristic including a parameter value for a first and a second predetermined percentile lower than 50 and for a third and a fourth predetermined percentile higher than 50;
at least one comparison module to compare the statistical characteristics of the distributions and selecting from said first and said second distribution as a first selected distribution the one having the higher parameter value for said first predetermined percentile and as a second selected distribution the one having the higher parameter value for said third predetermined percentile;
at least one range assignment module to: determine for the first selected distribution a parameter value associated with at least a second predetermined percentile lower than 50, to define at least a first value range having said parameter value as an upper bound and a second value range having said parameter value as a lower bound; to assign a partial prediction value to said value ranges, wherein a partial prediction value assigned to the first value range is a stronger indicator for said condition than a partial prediction value assigned to the second value range if the first selected distribution is the first distribution, and wherein a partial prediction value assigned to the first value range is a stronger indicator for the absence of said condition than a partial prediction value assigned to the second value range if the first selected distribution is the second distribution, and/or to define at least a third value range having the fourth parameter value of the second selected distribution as an upper bound and a fourth value range having said fourth parameter value as a lower bound, to assign a partial prediction value to said value ranges, wherein a partial prediction value assigned to the fourth value range is a stronger indicator for said condition than a partial prediction value assigned to the third value range if the second selected distribution is the first distribution, and wherein a partial prediction value assigned to the fourth value range is a stronger indicator for the absence of said condition than a partial prediction value assigned to the third value range if the second selected distribution is the second distribution;
wherein the definition of the value ranges and the assigned partial prediction values determine to which extent values for said indicative parameters determined for a particular individual contribute to the combined prediction value that indicates said condition or the absence thereof with said particular individual and that is obtained by adding the partial prediction values.

20. An indication apparatus for computing a combined prediction value indicative for the likelihood of a condition with an individual, the apparatus comprising,
a parameter value issuing module to issue for said individual respective individual values for a set of indicative parameters indicative for said condition, wherein respective indicative parameters are associated with respective parameter value ranges, including one or more of a pair of a first value range and a second value range, and pair of a third value range and a fourth value range,
a partial prediction value assignment module to determine for each of said indicative parameters which of the associated value ranges comprises the individual value for said indicative parameter, and to determine the partial prediction value for that associated value range,
said pair of a first value range and a second value range, and/or said pair of a third value range and a fourth value range and their associated partial prediction values being related to a respective first distribution of values for said indicative parameter in a control group which according to a Golden Standard does not have said condition and to a respective second distribution of values for said indicative parameter in a group of entities for which said condition is determined according to said Golden Standard, each of said distributions having a respective parameter value for a first percentile lower than 50, a second percentile lower than 50, a third percentile higher than 50, and a fourth percentile higher than 50, and wherein a first selected distribution selected from the first and the second distribution has a highest parameter value for said first percentile, and wherein a second selected distribution selected from the first and the second distribution has a lowest parameter value for said third percentile,
a combining module to determine the combined prediction value by combining, by adding, the partial prediction values obtained for each of the parameters,
wherein said first value range has the parameter value of the first selected distribution for the second percentile as an upper bound and a second value range has said parameter value as a lower bound,
wherein a partial prediction value assigned to the first value range contributes more to said combined prediction value predicting said condition than a partial prediction value assigned to the second value range if the first selected distribution is the first distribution, and wherein a partial prediction value assigned to the first value range contributes more to said combined prediction value predicting the absence of said condition than a partial prediction value assigned to the second value range if the selected distribution is the second distribution,
and/or wherein the third value range has the parameter value of the second selected distribution as an upper bound and the fourth value range has said parameter value as a lower bound, wherein a partial prediction value assigned to the fourth value range contributes more to said combined prediction value predicting said condition than a partial prediction value assigned to the third value range if the second selected distribution is the first distribution, and wherein a partial prediction value assigned to the fourth value range contributes more to said combined prediction value predicting the absence of said condition than a partial prediction value assigned to the third value range if the second selected distribution is the second distribution.

21. The indication apparatus according to claim 20, wherein the parameter value issuing module includes at least one reading unit for reading a value of a parameter from a storage unit.

22. The indication apparatus according to claim 20, wherein the set of parameters includes at least a biomarker, and wherein the parameter value issuing module includes at least one unit to determine a value for said biomarker in a urine or a serum sample of a person.

23. A computer-implemented indication method for computing a combined prediction value indicative for a condition of an individual, the method comprising,
determining with said individual respective individual parameter values for a set of indicative parameters indicative for said condition,
associating each of said indicative parameters with a pair of a first value range and a second value range, and/or a pair of a third value range and a fourth value range, each of the predetermined value ranges of a parameter being associated with a partial prediction value indicating the extent to which a parameter value in that predetermined value range is indicative for said condition,
said pair of a first value range and a second value range, and/or said pair of a third value range and a fourth value range and their associated partial prediction values being related to a respective first distribution of values for said indicative parameter in a control group which according to a Golden Standard does not have said condition and to a respective second distribution of values for said indicative parameter in a group of entities for which said condition is determined according to said Golden Standard, each of said distributions having a respective parameter value for a first percentile lower than 50, a second percentile lower than 50, a third percentile higher than 50, and a fourth percentile higher than 50, and wherein a first selected distribution selected from the first and the second distribution has a highest parameter value for said first percentile, and wherein a second selected distribution selected from the first and the second distribution has a lowest parameter value for said third percentile,
wherein said first value range has the parameter value of the first selected distribution for the second percentile as an upper bound and a second value range has said parameter value as a lower bound,
wherein a partial prediction value assigned to the first value range contributes more to said combined prediction value predicting said condition than a partial prediction value assigned to the second value range if the first selected distribution is the first distribution, and wherein a partial prediction value assigned to the first value range contributes more to said combined prediction value predicting the absence of said condition than a partial prediction value assigned to the second value range if the selected distribution is the second distribution,
and/or wherein the third value range has the parameter value of the second selected distribution as an upper bound and the fourth value range has said parameter value as a lower bound, wherein a partial prediction value assigned to the fourth value range contributes more to said combined prediction value predicting said condition than a partial prediction value assigned to the third value range if the second selected distribution is the first distribution, and wherein a partial prediction value assigned to the fourth value range contributes more to said combined prediction value predicting the absence of said condition than a partial prediction value assigned to the third value range if the second selected distribution is the second distribution.
determining for said individual for each of said parameters which of its associated predetermined value ranges comprises the determined individual parameter value for said parameter, and determining the partial prediction value for that associated predetermined value range,
for said individual determining the combined prediction value by combining by adding the partial prediction values obtained for each of the parameters.

24. The computer-implemented indication method according to claim 23, said combining further comprising modifying the partial prediction values obtained for each of the parameters by multiplication with a weighting factor.
